(19) United States
(12) Patent Application Publication
(10) Pub. No.: US 2022/0268760 A1
(43) Pub. Date: Aug. 25, 2022

(54) ATRIAL CARDIAC MICROTISSUES FOR CHAMBER-SPECIFIC ARRHYTHMOGENIC TOXICITY RESPONSES

(71) Applicants: Brown University, Providence, RI (US); Rhode Island Hospital, Providence, RI (US)

(72) Inventors: Kareen L. K. COULOMBE, Pawtucket, RI (US); Bum-Rak CHOI, Warwick, RI (US); Arvin H. SOEPRIATNA, Pawtucket, RI (US); Tae Yun KIM, Decatur, GA (US); Mark C. DALEY, Providence, RI (US)

(21) Appl. No.: 17/675,895

(22) Filed: Feb. 18, 2022

Related U.S. Application Data
(60) Provisional application No. 63/151,399, filed on Feb. 19, 2021.

(51) Int. Cl. G01N 33/50; C12N 5/071; C12N 5/077; C12N 5/074; G01N 15/14; C12N 13/00

(52) U.S. Cl. CPC G01N 33/5014; C12N 5/0697; C12N 5/0657; C12N 5/0696; G01N 33/5088; G01N 33/5061; G01N 15/14; C12N 13/00; C12N 2513/00; C12N 2506/45; C12N 2501/415; G01N 2015/1006

(57) ABSTRACT

The invention provides a robust in vitro 3D atrial tissue platform made from human induced pluripotent stem cell (hiPSC)-derived cardiomyocytes. The platform is useful for evaluating atrial-specific chemical responses experimentally and computationally.

### REFERENCE TO RELATED APPLICATIONS

This invention is related to provisional patent application U.S. Ser. No. 63/151,399, filed Feb. 19, 2021, the contents of which are hereby incorporated by reference.

### TECHNICAL FIELD OF THE INVENTION

This invention generally relates to an apparatus for growing cells or for obtaining fermentation or metabolic products, i.e., bioreactors or fermenters specially adapted for specific uses for producing artificial tissue or for ex-vivo cultivation of tissue. This invention also relates to tissue engineering, three dimensional models, hiPSC-derived cardiomyocytes, optical mapping, computational modeling, and data analysis.

### BACKGROUND OF THE INVENTION

Atrial fibrillation (AFib) is the most common form of sustained cardiac arrhythmia. Atrial fibrillation has become increasingly prevalent globally, with the number of U.S. cases projected to double between the years 2010 and 2030. Colilla et al., Am. J. Cardiol., 112, 1142-1147 (2013). Developing long-lasting treatments for atrial fibrillation is important for reducing the risk of stroke and heart failure in our aging population. Kornej et al., Circ. Res., 127, 4-20 (2020); Morillo et al., J. Geriatr. Cardiol ,14, 195-203 (2017).

There are currently two accepted treatments for atrial fibrillation: radiofrequency ablation and antiarrhythmic drugs. For radiofrequency ablation, accurate identification of all ablation targets remains challenging. Up to 20% of atrial fibrillation cases recur post-ablation therapy. Gaztanaga et al., Heart Rhythm, 10, 2-9 (2013); Mujovic et al., Adv. Ther., 34, 1897-1917 (2017); Rottner et al., Cardiol. Ther., 9, 45-58 (2020). For class I and III antiarrhythmic drugs, which respectively block sodium (Na+) and potassium (K⁺) channels to restore sinus rhythm, while these drugs are moderately effective at suppressing the disease, they indiscriminately target both the atria and ventricles, increasing the patient's susceptibility to developing potentially fatal ventricular arrhythmias via QT prolongation, particularly when class III K⁺ channel blockers are prescribed. Woods & Olgin, Circ. Res., 114, 1532-1546 (2014). Recent drug discovery efforts for atrial fibrillation treatment have focused on developing atrial selective drugs that target ion channels primarily expressed in the atria, such as the ultrarapid delayed rectifier K⁺ current, I_{Kur} Ravens & Wettwer, Cardiovasc. Res., 89, 776-785 (2011); Tamargo, Caballero, Gomez, & Delpon, Expert Opin. Investig, Drugs, 18, 399-416 (2009). The dose-dependent response on action potential (AP) property, efficacy, and safety of several I_{Kur} blockers that under development require further investigation. Dan & Dobrev, Int. J. Cardiol. Heart Vasc., 21, 11-15 (2018); Hanley, Robinson, & Kowey, Circ. Arrhythm. Electrophysiol., 9, e002479 (2016).

To fully guide the development of atrial-specific drugs and evaluate their safety, there is a need in the cardiovascular art for robust in vitro screening assays for cardiotoxic assessment. The United States Food & Drug Administration's Comprehensive In Vitro Proarrhythmia Assay (CiPA) initiative of 2013 emphasized the need to integrate human platforms into the drug development process. Pang et al., Circ. Res., 125, 855-867 (2019). In vivo animal models often fail to replicate the human drug response due to species-specific differences in ion channel expression levels. Tanner & Beeton, Front. Biosci. (Landmark ed.), 23, 43-64 (2018). Off-target drug effects which may alter the activity of multiple ion channels must be thoroughly characterized to monitor for unexpected pro-arrhythmic hazards.

Many research groups use human induced pluripotent stem cell-derived cardiomyocytes (hiPSC-CMs) for toxicity screening of therapeutics, because they recapitulate key physiological properties, including proper human ion channel expression levels, contractility, and action potential shape. Gintant et al., Circ. Res., 125, e75-e92 (2019); Sinnecker, Laugwitz, & Moretti., Pharmacol. Ther., 143, 246-252 (2014). Many hiPSC-cardiomyocyte cardiotoxicity studies to date relied upon homotypic two-dimensional (2D) monolayer cultures. These cultures do not account for the complex 3D cell-to-cell interactions between multiple cell types, which are now known to modulate the electrophysiological behavior of tissues. Sacchetto et al., Int. J. Mol. Sci., 21 (2020).

Only recently has there been a paradigm shift to using 3D microtissues or macrotissues to investigate drug effects on diverse pro-arrhythmic metrics, such as action potential properties and calcium transients. But many 3D microtissues or macrotissues are still challenged with issues related to limited throughput.

The cardiotoxicity field of the cardiovascular art remains focused on ventricular responses to potentially cardiotoxic drugs, whose effects on atrial arrhythmogenicity are often overlooked. Inherent differences in ion channel expressions between the atria and ventricles, as well as the presence of atrial-specific channels, may result in chamber-specific arrhythmogenic responses to the same compound. Grandi et al., Circ. Res., 109, 1055-1066 (2011); Schram, Pourrier, Melnyk, & Nattel, Circ. Res., 90, 939-950 (2002); Walden, Dibb, & Trafford, J. Mol. Cell Cardiol., 46, 463-473 (2009). While ventricular arrhythmias tend to be more deadly than their atrial counterparts, a thorough characterization of chamber-specific responses is imperative in establishing high safety standards for drug testing across all patient populations, including in patients with Wolff-Parkinson-White (WPW) syndrome, who develop accessory electrical conduction pathway between the atrium and ventricle. Centurion, J. Atr. Fibrillation, 4, 287 (2011).

Although atrial fibrillation is the most prevalent disorder of electrical conduction, the mechanisms behind atrial arrhythmic toxicity remain elusive. Taken together, there remains a need in the cardiovascular art to accurately assess the cardiotoxic effects of novel therapeutics in both atrial and ventricular cell populations with high throughput.

### SUMMARY OF THE INVENTION

The invention provides a robust in vitro three-dimensional (3D) atrial tissue platform made from human induced pluripotent stem cell (hiPSC)-derived cardiomyocytes. The platform usefully provides an atrial in vitro platform that enables chamber-specific evaluation of arrhythmic risk. The platform is also useful for evaluating atrial-specific chemical responses experimentally and computationally.

In a first embodiment, the invention provides a highly sensitive and predictive in vitro screening platform. The platform comprises 3D atrial and ventricular microtissues self-assembled from hiPSC-cardiomyocytes. The inventors showed that high-purity cardiomyocyte (>75% cTnT⁺) demonstrated (1) subtype specification by MLC2v⁺, as reflected in (2) shortened action potential duration (APD) and (3) spontaneous action potential activity in atrial microtissues compared to ventricular microtissues. Atrial-specific responses to 4-aminopyridine (Iₜₒ and I_{Kur} blocker) are detected in the ventricular microtissues. Atrial-specific responses to ivabradine (I_{f} blocker) are not detected in the ventricular microtissues.

The inventors tested drugs that specifically target ion channels. Because atrial and ventricular subtypes are differentially expressed, the inventors showed through gene expression data in FIG. 8B and TABLE 6 that atrial and ventricular microtissues share many major ion channels important in healthy cardiac electrophysiology.

When persons having ordinary skill in the art conduct drug screening using drugs that target these shared ion channels, they observe unique electrophysiological responses between atrial and ventricular microtissues due to compensation effects driven by the presence of other ion channels that are unique to atrial or are shared but differentially expressed between the two subtypes. The inventors showed that microtissues exhibit a more mature phenotype with longer three-dimensional (3D) culture time. See the ion channel gene expression data in FIG. 8B.

When persons having ordinary skill in the art use GCaMP6f expressing human induced pluripotent stem cell line, they can look at Ca²⁺ transient traces in addition to voltage signals. The microtissues can visibly be seen to beat. Thus, the microtissues are useful for studying contractility and tissue force mechanics.

In a second embodiment, the invention provides a method of making a highly sensitive and predictive in vitro screening platform. See EXAMPLE 1 and EXAMPLE 2. The inventors differentiated atrial and ventricular cardiomyocytes (aCMs/vCMs) from GCaMP6f-expressing hiPSCs by Wnt modulation with or without the addition of retinoic acid, followed by metabolic-based lactate purification and flow cytometry to assess purity and subtype. The inventors thus generated self-assembling 3D atrial and ventricular microtissues from hiPSC-cardiomyocytes, assessed their calcium transients, and used optical mapping to characterize cardiomyocyte subtype differences in action potential properties. The inventors performed GCaMP fluorescence imaging to measure spontaneous action potential activity. Self-assembling 3D microtissues formed with cardiomyocytes and 5% human cardiac fibroblasts in agarose microwells. The inventors electrically stimulated the 3D microtissues for one week before high resolution action potential (AP) optical mapping. Action potential responses to atrial-specific drugs were quantified across physiologically relevant doses.

In a third embodiment, the inventors modified ion channel conductances from a published hiPSC-cardiomyocyte computational model by Paci et al., Ann. Biomed. Eng., 41, 2334-2348 (2013) to mimic action potential waveforms in these atrial and ventricular cardiomyocyte microtissues. Increased specificity of the atrial model incorporates atrial specific currents I_{Kur} to replicate experimental dose responses. The inventors modified ion channel conductancesfrom the Paci model to match the electrophysiology of the microtissue data produced from these assays, to an atrial specific ion channel that the Paci model was missing based.

In a fourth embodiment, the invention provides a method of evaluating atrial-specific arrhythmogenic toxicity responses by atrial specific metrics of spontaneous beating cycle length, pacemaker potential and action potential amplitudes, action potential rise time, APD₃₀, APD₅₀, APDₜᵣᵢ, and APDₘₐₓ with high throughput and automatic signal processing routines.

The inventors validated the highly sensitive and predictive in vitro screening platform by evaluating chamber-specific responses to atrial specific drugs targeting the I_{Kur} and I_{f} channels. The inventors used the findings to update the previously published hiPSC-cardiomyocyte action potential computational model by Paci et al., Ann. Biomed. Eng., 41, 2334-2348 (2013), to include the atrial specific channel, I_{Kur}

The in vitro platform for screening atrial toxicants provided by the invention is both robust and sensitive, with high throughput, enabling studies focused at elucidating the mechanisms underlying atrial arrhythmias.

The in vitro platform for screening atrial toxicants provided by the invention is robust and sensitive, with high throughput, enabling studies focused at elucidating the mechanisms underlying atrial arrhythmias.

The inventors demonstrated an in vitro screening platform for chamber-specific evaluation of arrhythmogenic toxicity responses using human atrial 3D cardiac microtissues. Using GCaMP fluorescence imaging and optical mapping, the inventors highlighted important differences in the spontaneous activity, calcium handling, and action potential properties of atrial and ventricular microtissues. The inventors then detected dose-dependent and chamber-specific responses to the atrial-selective drug 4-aminopyridine (4-AP), as well as ivabradine, stressing the importance of incorporating both atrial and ventricular toxicity assessment in the development of novel therapeutics. The inventors used the action potential traces to incorporate the atrial-sensitive I_{Kur} current into an established hiPSC-cardiomyocyte action potential computational model²⁴ and investigate differences in the behaviors of major ion channels between the two microtissue subtypes.

The cardiomyocyte subtype differences in spontaneous beating rates, calcium handling, and action potential properties measured in the cultures (FIG. 2 and FIG. 3) are consistent with those reported in the literature. In agreement with the findings of other groups, Cyganek et al., JCI Insight, 3 (2018); Pei et al., Stem Cell Res 19: 94-103 (2017). hiPSC-atrial cardiomyocytes exhibit reduced MLC2v expression (FIG. 6) and develop faster spontaneous beating rates compared to hiPSC-ventricular cardiomyocytess. This difference is likely caused by increased HCN4 and decreased KCNJ2 expressions in hiPSC-atrial cardiomyocytes, which respectively are responsible for regulating diastolic depolarization through the I_{f} current and establishing stable resting membrane potential via the I_{K1} current. Garg et al., Circ. Res. 123, 224-243 (2018). This behavior is shown in the action potential traces (FIG. 3A), where a positively drifting baseline potential was observed in between 1 Hz pacing in atrial, but not in ventricular microtissues, and further recapitulated by the reduced I_{K1} and increased 4 currents of the atrial hiPSC-cardiomyocyte computational model (FIG. 5C). Interestingly, longer culture times increased spontaneous activity in atrial cardiomyocytes, while remaining unchanged in ventricular cardiomyocytes, even though both subtypes are physiologically quiescent when fully matured. Differences in maturation progression may hallmark between atrial cardiomyocytes and ventricular cardiomyocytes, with increased I_{K1} expression in ventricular cardiomyocytes suppressing automaticity at an earlier timepoint than atrial cardiomyocytes.

The atrial action potential traces showed a triangulated profile, as opposed to the “spike-and-dome” shaped ventricular action potential with a prominent plateau phase, like the literature on adult human cardiomyocytes. Garg et al., Circ. Res. 123, 224-243 (2018). These traces are attributed to well-known differences in Ca²⁺ handling between the two cardiomyocyte subtypes, with atrial cardiomyocytes exhibiting smaller systolic calcium transients that decayed more rapidly. Walden, Dibb, & Trafford, J. Mol. Cell Cardiol., 46, 463-473 (2009). This difference was observed in the calcium transients traces in 2D monolayer cultures (FIG. 2A). Although rate-dependent changes in calcium transients duration, the calcium transients of ventricular cardiomyocytes still demonstrated wider peaks with a plateau phase, suggesting improved calcium handling that prolongs action potential duration (FIG. 2 and FIG. 3). The slow rise time in atrial microtissues corroborates findings of slowed upstroke velocity reported in atrial tissues by several groups. Goldfracht et al., Nature Commun., 11, 75 (2020). The slow rise time is likely driven by a more depolarized and drifting resting membrane potential partially inactivating sodium channels available for initiating successful action potential generation.

### BRIEF DESCRIPTION OF THE DRAWINGS

For illustration, some embodiments of the invention are shown in the drawings described below. Like numerals in the drawings indicate like elements throughout. The invention is not limited to the precise arrangements, dimensions, and instruments shown.

Cardiac directed differentiation and 3D microtissue generation. FIG. 1 shows the design of the platform. FIG. 1A is a timeline overview of cardiomyocyte differentiation to 3D microtissue formation. Cardiac-directed differentiation was achieved via timed modulation of the Wnt signaling pathway. Atrial-subtype specification was obtained by retinoic acid (RA) supplementation. FIG. 1B comprises a pair of photographic images showing that beating cardiomyocytes were lactate-purified before being used to assess spontaneous action potential (AP) activity via GCaMP fluorescence imaging. FIG. 1C is a drawing that shows self-assembling 3D microtissues were generated by seeding hiPSC-cardiomyocytes into agarose hydrogel molds, followed by electrical stimulation. FIG. 1D shows action potential traces captured with optical mapping to assess changes in action potential properties in response to different drug treatments.

Cardiac subtype, maturation state, and 3D environment influence spontaneous beating rates. FIG. 2 shows differences in spontaneous action potential activity firing rates and Ca²⁺ transients between atrial and ventricular subtypes and across 2D and 3D structures. FIG. 2A shows GCaMP traces of hiPSC-cardiomyocytes from 2D culture. FIG. 2B shows the results of these GCaMP traces, showing significant differences in spontaneous action potential firing rates with subtype and age. Atrial hiPSC-cardiomyocytes demonstrated faster automaticity than their ventricular counterpart and continued to exhibit faster pacing with longer culture times. FIG. 2C shows GCaMP traces from 3D culture. The incorporation of 5% human cardiac fibroblast to generate 3D microtissues reduced automaticity in atrial samples, while ceasing spontaneous activity in ventricular samples. FIG. 2D shows the results of these GCaMP traces. Ventricular hiPSC-cardiomyocytes experienced longer durations of Ca²⁺ handling likely attributed to slower spontaneous activity rates. Values are shown as mean±standard deviation (*p<0.05, ***p<0.001, ****p<0.0001). denotes statistically significant differences with every other conditions.

Action potential properties of atrial vs. ventricular microtissues. FIG. 3 shows differences in action potential properties between atrial and ventricular 3D microtissues under 1 Hz pacing. FIG. 3A shows several action potential traces. The comparison of action potential traces showed shorter action potential duration (APD) in atrial microtissues, with longer culture resulting in a more prominent sharp Iₜₒ peak (green line, black arrow). FIG. 3B shows that atrial microtissues exhibited significantly slower rise time when compared to ventricular microtissues and shorter FIG. 3C APD₃₀, FIG. 3D APD₅₀, FIG. 3E APD₈₀, and FIG. 3F APD_{MxR}. FIG. 3G shows that APD_{Tri} remained unchanged. Values are shown as mean±standard deviation (****p<0.0001).

4-Aminopyridine prolongs action potential duration in atrial cardiomyocyte microtissues. Ivabradine reduces spontaneous beating rates. FIG. 4 shows dose-dependent effects of 4-aminopyridine (4-AP) and ivabradine on action potential properties. FIG. 4A shows that 4-aminopyridine treatment resulted in dose-dependent action potential duration prolongation, as observed by the rightward shift of the APD₈₀ cumulative probability distribution. FIG. 4B shows that these effects were not observed in the ventricular samples. FIG. 4C shows that treatment with ivabradine across all tested dosages significantly reduced spontaneous action potential events. In a small subset of microtissues with spontaneous activity, events resembling failed depolarizations are seen (blue arrow and blue boxes). Values are shown as mean±standard deviation (*p<0.05, **p<0.01, ****p<0.0001).

Computational model of hiPSC-cardiomyocytes. FIG. 5 shows a computational modeling of hiPSC-cardiomyocyte action potentials. Comparison between modeled and experimentally obtained action potential traces for FIG. 5A atrial and FIG. 5B ventricular microtissues demonstrate a good fit. FIG. 5C shows that modeled traces of the major ion currents responsible for determining action potential shape demonstrate variations in activation/inactivation kinetics and current intensities between the atrial and ventricular hiPSC-cardiomyocytes. hiPSC-atrial cardiomyocytes exhibit smaller I_{Na}, I_{CaL}, I_{Kr}, I_{Ks}, and I_{K1}, but more prominent Iₜₒ and I_{f}, similar to findings reported in the literature for adult cardiomyocytes. Atrial specific I_{Kur} channel was incorporated into the computational model. FIG. 5D is a line graph showing modeled changes in APD₃₀, APD₅₀, and APD₈₀ values and FIG. 5E atrial action potential waveforms in response to changes in I_{Kur} conductances, g_{Kur}.

FIG. 6 shows 3D microtissue compaction. FIG. 6A is a set of representative images of 3D cardiac microtissues with 5% human cardiac fibroblasts undergoing tissue compaction over eight days of electrical stimulation. FIG. 6B is a line graph showing a quantitative evaluation of microtissue diameters showed significant compaction within the first three days of tissue formation, reaching average diameters of 387.65±1.0 μm in atrial and 374±6.8 μm in ventricular microtissues by day 8.

FIG. 7 shows flow cytometry of cardiomyocyte purity and MLC2v expression. FIG. 7A and FIG. 7B shows generated and selected 3D microtissues, which the inventors could consistently generate using hiPSC-a/vCMs with >75% cardiomyocyte purity (cTnT⁺). FIG. 7C and FIG. 7D are a pair of dot plots of cTnT and MLC2v expression, showing significantly higher MLC2v expression in ventricular samples compared to atrial samples FIG. 7F, although overall MLC2v expression remained low. These low MLC2v expressions were likely attributed to the maturation state of the hiPSC-cardiomyocytes. FIG. 7E shows that lactate purification significantly improved cardiomyocyte purity. Values are shown as mean±standard deviation (*p<0.05, ****p<0.0001).

FIG. 8 shows the relative gene expression of 3D cardiac microtissues. FIG. 8A is a bar graph of a qPCR analysis showing reduced expression levels of ventricular markers (MYL2 and IRX4) and increased expression levels of atrial markers (NR2F2 and NPPA) in 3D atrial microtissues compared to ventricular microtissues. FIG. 8B is a bar graph of a qPCR analysis of select ion channels showing a significant increase in KCNA5 gene expression, associated with the atrial specific I_{Kur} channel, and a modest increase in KCNJ3 gene expression, associated with the atrial specific I_{K,ACh} channel, in atrial microtissues compared to ventricular microtissues. Interestingly, no differences in HCN4 gene expression, associated with the I_{f} channel, was detected between atrial and ventricular microtissues, although the inventors observed a decrease in KCNJ2 gene expression, associated with the I_{K1} channel responsible for establishing resting membrane potential. Values are plotted relative to gene expression of ventricular microtissues (y=0 line), shown as mean±standard deviation (*p<0.05, **p<0.01), and represent data averaged from n=3 differentiation batches.

FIG. 9 shows the reproducibility of 3D microtissue measurements. Averaged action potential waveforms from representative FIG. 9A atrial and FIG. 9B ventricular 3D microtissues across multiple beats demonstrated minimal beat-to-beat variation, as shown by the narrow 95% confidence interval (gray shades). Averaged action potential waveform across five randomly selected microtissues within the same differentiation batch demonstrated minimal variability in action potential metrics between FIG. 9C atrial microtissues, although some variability was observed between FIG. 9D ventricular microtissues during the repolarization phase. FIG. 9E and FIG. 9F show a comparison of APD₃₀, APD₅₀, and APD₈₀ showed batch-to-batch differences, but these variations were small and remained distinct between atrial and ventricular microtissues. Values are shown as mean±standard deviation. Asterisks' colors correspond to parameter showing statistical significance, with gray, red, and blue corresponding to APD₃₀, APD₅₀, and APD₈₀ respectively (*p<0.05. **p<0.01).

FIG. 10 show a dose-dependent effects of 4-aminopyridine (4-AP) on several action potential properties. The dose-dependent effects of 4-aminopyridine on rise time, APD₃₀, APD₅₀, APD₈₀, APD_{MxR}, and APD_{Tri}/APD_{MXR} are summarized for both atrial and ventricular 3D microtissues. Values are shown as mean±standard deviation (*p<0.05, **p<0.01, ***p<0.001, ****p<0.0001) and represent data from n=3 differentiation batches.

FIG. 11 shows ivabradine-induced loss of spontaneous activity. FIG. 11A shows traces from a small subset of spontaneous atrial microtissue treated with 5 μM Ivabradine underwent events resembling failed depolarization (black arrows) that was recovered with 1 Hz electrical pacing. FIG. 11B is a bar graph showing that higher dosages of ivabradine resulted in loss of spontaneous activity.

FIG. 12 is set of traces showing that APDₘₐₓ measures APD through detecting the maximum hyperpolarization point.

### DETAILED DESCRIPTION OF THE INVENTION

### Industrial Applicability

The cardiotoxicity field focuses primarily on ventricular responses to drugs while their effects on atrial electrophysiology remain understudied, due partly to the lack of available atrial testing platforms with high throughput and validated responses to atrial-targeting drugs. In vitro screening platforms using hiPSC-cardiomyocytes have proven to be invaluable for cardiotoxic assessment of novel therapeutics. However, the debate surrounding the appropriate maturation timepoint of hiPSC-cardiomyocytes and environmental factors necessary to accurately model human in vivo responses to drugs without impeding throughput remains heated.

The in vitro platform for screening atrial toxicants provided by the invention is both robust and sensitive, with high throughput, enabling studies focused at elucidating the mechanisms underlying atrial arrhythmias.

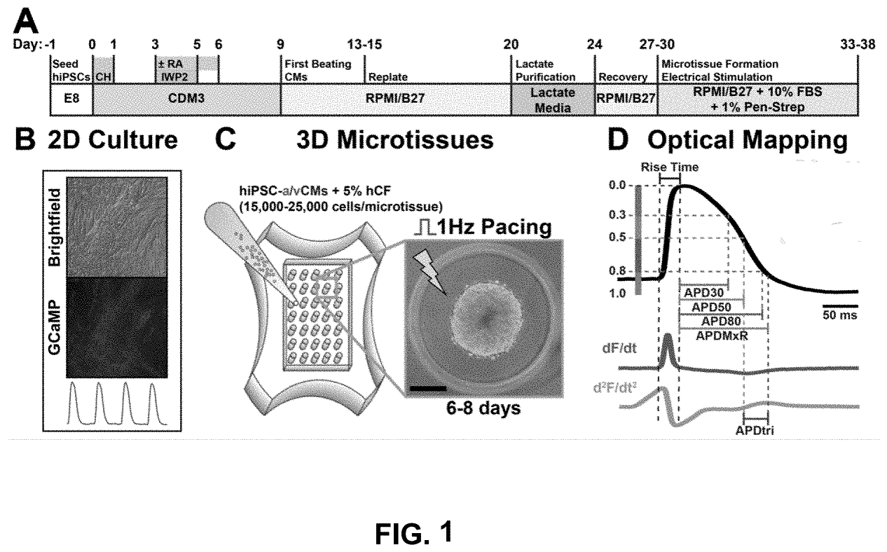

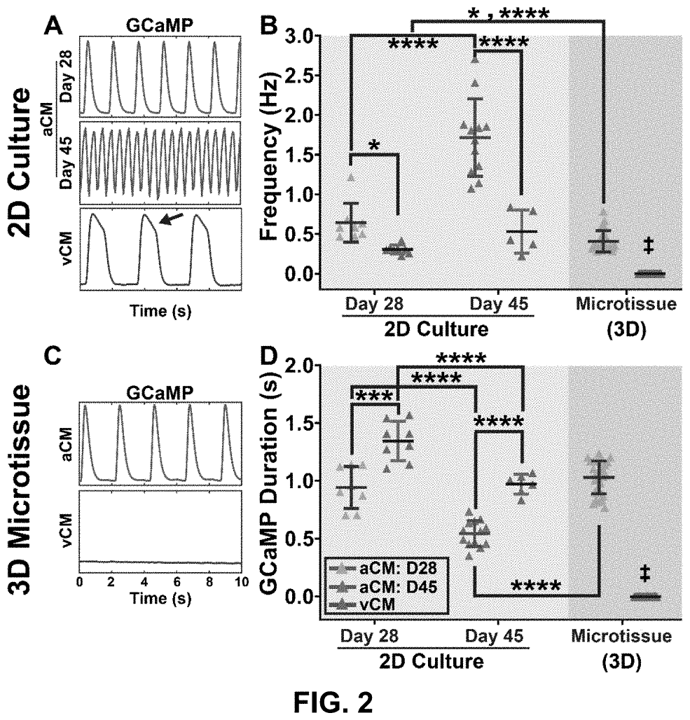

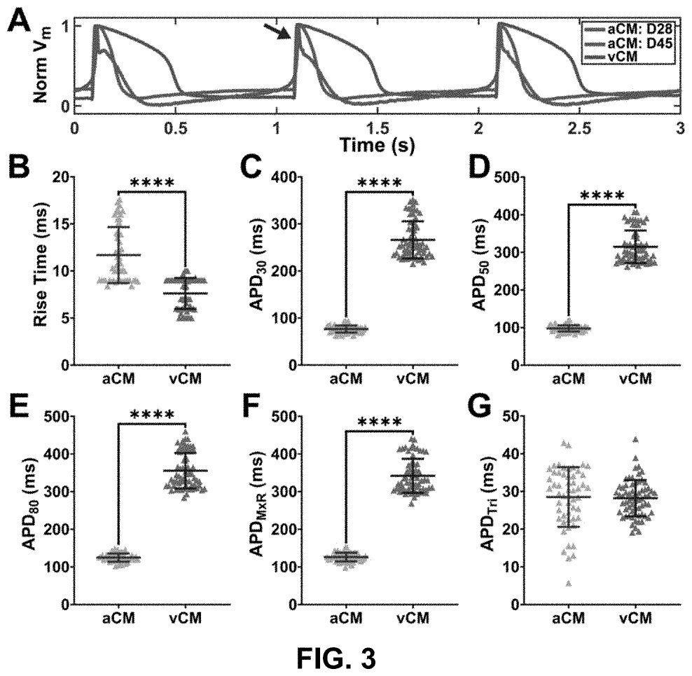

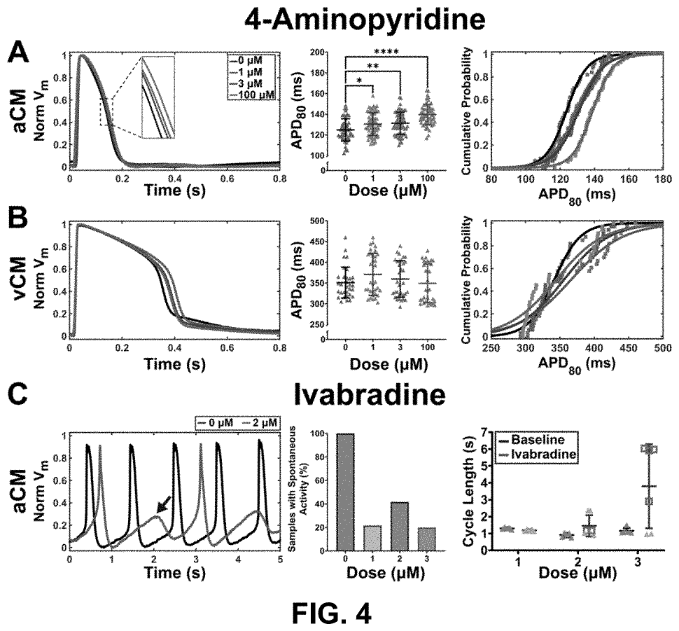

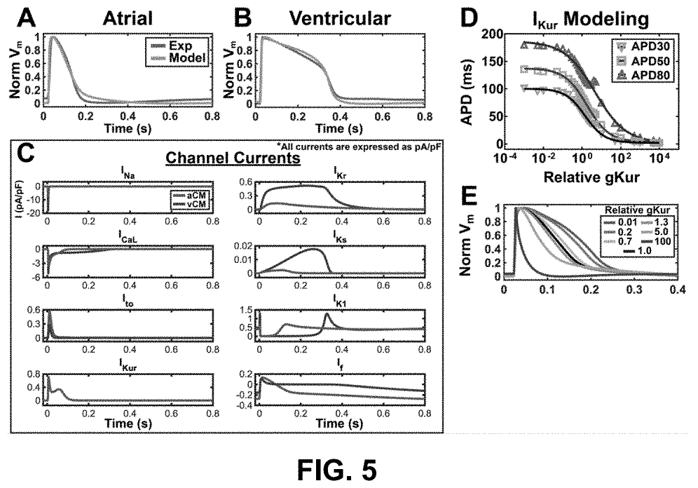

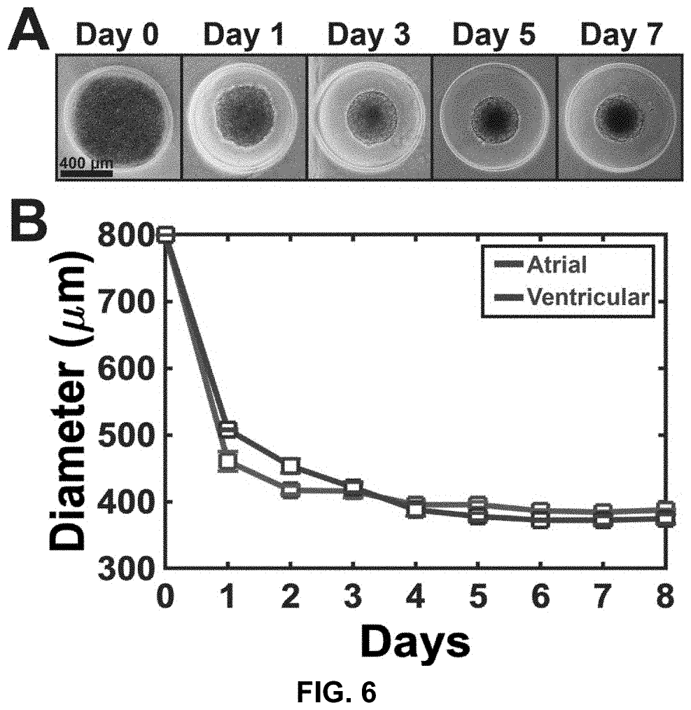

The advantages of the atrial platform result from a decision to lactate-select the input cardiomyocytes for improved purity (FIG. 6) and to rely on the natural process of cardiomyocyte self-assembly to generate 3D microtissue interspersed with human cardiac fibroblasts, without the use of an unnatural substrate. The rationale is to recapitulate the microenvironment of the native myocardium, which includes a highly organized 3D cardiomyocyte arrangement and heterocellular cross-talk with fibroblasts, the most prevalent non-cardiomyocyte population in the myocardium. Zhou & Pu, Circ. Res. 118, 368-370 (2016). 3D assembly has been shown to accelerate cardiomyocyte maturation rate' while improving action potential propagation due to increased cell-to-cell connectivity. Sacchetto et al., Int. J. Mol. Sci., 21 (2020). The fibroblasts function of modulating the electrophysiological properties of tissues is becoming widely accepted. Zhang, Su, & Mende, Am. J. Physiol. Heart Circ. Physiol., 303, H1385-1396 (2012). The inventors previously reported that the addition of 5% human cardiac fibroblasts optimally improves electromechanical function and promotes compaction in the engineered tissues. Kofron et al., Sci. Reports, 11(1),10228 (2021); Rupert, Kim, Choi, & Coulombe, Stem Cells Int., 2020, 9363809 (2020). The inventors demonstrated that cardiomyocyte reassembly to 3D in the presence of fibroblasts significantly decreased spontaneous beating rates in atrial microtissues while eliminating spontaneous activity in ventricular microtissues (FIG. 2), which is explained by cardiomyocyte-fibroblast coupling elevating the resting membrane potential of cardiomyocyte to inactivate sodium channels and increase excitation threshold. Jacquemet & Henriquez, Europace, 9 Suppl 6, vi29-37 (2007). The inventors matured the 3D microtissues for a minimum of six days under electrical stimulation to improve electromechanical function. Radisic et al., Proc. Natl. Acad. Sci., U.S.A., 101, 18129-18134 (2004). They showed in a small subset of the samples that increasing 2D culture times to 45-days further promote maturation in the atrial cardiomyocytes, resulting in a pronounced Iₜₒ peak that is more reflective of adult human atrial cardiomyocytes (black arrow, FIG. 3A). Thus, the platform can easily be adapted to better reproduce in vivo action potential behaviors of adult humans, by longer 2D or 3D culture, despite the current set-up proving sufficient in detecting dose-dependent and chamber specific responses to atrial-selective drugs. The optical mapping approach, which averages the behaviors of all the cells within a single microtissue to reconstruct a representative action potential trace, presents a robust method to characterizing drug responses highly quantitative action potential metrics of bulk tissue behavior that are highly reproducible with reduced variability.

### Definitions

For convenience, the meaning of some terms and phrases used in the specification, examples, and appended claims, are listed below. Unless stated otherwise or implicit from context, these terms and phrases shall have the meanings below. These definitions aid in describing particular embodiments but are not intended to limit the claimed invention. Unless otherwise defined, all technical and scientific terms have the same meaning as commonly understood by a person having ordinary skill in the art to which this invention belongs. A term's meaning provided in this specification shall prevail if any apparent discrepancy arises between the meaning of a definition provided in this specification and the term's use in the biomedical art.

4-Aminopyridine (4-AP, fampridine, dalfampridine, Ampyra™, Fampyra™) is an organic molecule with the chemical formula C₅H₄N—NH₂. CAS Registry Number is 504-24-5. The molecule is one of the three isomeric amines of pyridine. 4-Aminopyridine is an Iₜₒ and I_{Kur} blocker. The molecule It is used as a research tool in characterizing subtypes of the potassium channel. This molecule is commercially available.

Action potential (AP) has the cardiovascular art-recognized meaning. In physiology, an action potential occurs when the membrane potential of a specific cell location rapidly rises and falls. This depolarization then causes adjacent locations to similarly depolarize. Action potentials occur in several types of animal cells, called excitable cells, including cardiomyocytes.

Action potential duration (APD) has the cardiovascular art-recognized meaning. The cardiac action potential is a brief change in voltage (membrane potential) across the cell membrane of heart cells. This is caused by the movement of charged atoms (called ions) between the inside and outside of the cell, through proteins called ion channels. Cardiac action potentials in the heart differ from action potentials found in neural and skeletal muscle cells. In a typical nerve, the action potential duration is about one millisecond. In skeletal muscle cells, the action potential duration is approximately 2-5 milliseconds. By contrast, the duration of cardiac action potentials ranges from 200 to 400 milliseconds. Unlike the action potential in skeletal muscle cells, the cardiac action potential is not initiated by nervous activity. Instead, the action potential arises from a group of specialized cells, that have automatic action potential generation capability.

Atrial cardiomyocyte (aCM) has the cardiovascular art-recognized meaning. Atrial and ventricular cardiomyocytes form the muscular walls of the heart (the myocardium). Atrial myocytes have a different ultrastructure compared to ventricular myocytes. They have differential gene expression patterns regarding, e.g., transcription factors, structural proteins, and ion channels. Ng, Wong, & Tsang, Differential gene expressions in atrial and ventricular myocytes: insights into the road of applying embryonic stem cell-derived cardiomyocytes for future therapies. Am. J. Physiol. Cell Physiol., 299(6), C1234-49 (December 2010). They also display distinct functions.

Atrial fibrillation (AFib) has the cardiovascular art-recognized meaning.

CACNA1C has the cardiovascular art-recognized meaning of a gene (Hs00167681_m1) that codes for the ion channel I_{CaL} (calcium channel, voltage-dependent, L type, alpha 1C subunit).

CACNA1D has the cardiovascular art-recognized meaning of a gene (Hs00167753_m1) that codes for the ion channel I_{CaL} (atrial subunit) (calcium channel, voltage-dependent, L type, alpha 1D subunit).

Cardiac troponin T (cTnT) has the cardiovascular art-recognized meaning and is an early marker of acute myocardial infarction.

Cardiomyocyte has the cardiovascular art-recognized meaning as the contractile cells of the cardiac muscle. The cell is striated, containing thick and thin proteins arranged linearly. These filaments are composed, like other striated muscle cells, largely of actin and myosin. The cell has an abundant supply of mitochondria that supply the energy needed by the cell for regular muscular contraction. Cardiomyocytes are the contracting cells that allow the heart to pump.

GCaMP6 has the cardiovascular art-recognized meaning of a synthetic genetically encoded calcium indicator that is a synthetic fusion of green fluorescent protein (GFP), calmodulin (CaM), and M13, a peptide sequence from myosin light-chain kinase. GCaMP6 fluorescent indicator proteins enable reliable detection of single action potential responses in vivo and facilitate the measurement of synaptic calcium signals. Many GCaMP6 variants are commercially available. See also Chen et al., Ultrasensitive fluorescent proteins for imaging neuronal activity. Nature, 499(7458):295-300 (Jul. 18, 2013).

GCaMP6f has the cardiovascular art-recognized meaning of a synthetic genetically encoded calcium indicator that is a synthetic fusion of green fluorescent protein (GFP) derived from Aequorea victoria, calmodulin (CaM), and M13, a peptide sequence from myosin light-chain kinase. GCaMP6f, had faster rise time and a faster decay time than other GCaMP6 variants. GCaMP6f is commercially available. See also Chen et al., Ultrasensitive fluorescent proteins for imaging neuronal activity. Nature, 499(7458):295-300 (Jul. 18, 2013).

HCN4 has the cardiovascular art-recognized meaning. Potassium/sodium hyperpolarization-activated cyclic nucleotide-gated channel 4 (HCN4; Hs00923522 m1) encodes the HCN4 channels responsible for the hyperpolarization-activated funny current (I_{f}) essential to sinoatrial node automaticity.

Human induced pluripotent stem cell (hiPSC) has the cardiovascular art-recognized meaning. A hiPSC is a body cell that has been reprogrammed to behave like an embryonic stem cell and be able to differentiate into cells that could regenerate and repair many kinds of damaged or diseased tissues.

Human induced pluripotent stem cell-derived myocyte (hiPSC-CM) has the cardiovascular art-recognized meaning. In addition to being derivable from human induced pluripotent stem cells, human induced pluripotent stem cell-derived myocyte are commercially available.

Ion channel conductance has the cardiovascular art-recognized meaning.

IRX4 has the cardiovascular art-recognized meaning. The Iroquois homeobox 4 (IRX4; Hs00212560_m1) appears to have several during pattern formation of vertebrate embryos. IRX4 is a ventricular marker.

Ivabradine (Corlanor™; Procoralan™) (CAS Number 155974-00-8) is an I_{f} blocker. Ivabradine is in a class of medications called hyperpolarization-activated cyclic nucleotide-gated (HCN) channel blockers. It works by slowing the heart rate so the heart can pump more blood through the body each time it beats. Ivabradine is used to treat certain adults with heart failure to decrease the risk that their condition will worsen and need to be treated in a hospital. It is also used to treat a certain type of heart failure in children six months of age and older due to cardiomyopathy. Ivabradine is commercially available.

IWP2 (Cas Number 686770-61-6) is a Wnt production inhibitor that is commercially available from Tocris Bioscience, Minneapolis, Minn., USA and other sources.

KCNA5 has the cardiovascular art-recognized meaning. The potassium voltage-gated channel, shaker-related subfamily, member 5 (KCNAS; Hs00969279_s1) encodes the atrial specific I_{Kur} channel.

KCND3 has the cardiovascular art-recognized meaning. Potassium voltage-gated channel subfamily D member 3 (KCND3; Hs00542597_m1) encodes the Iₜₒ channel.

KCNH2 has the cardiovascular art-recognized meaning. KCNH2 (hERG, the human Ether-a-go-go-Related Gene, Hs00165120_m1) encodes a protein known as Kᵥ11.1, the alpha subunit of a potassium ion channel. This ion channel (‘hERG’) contributes to the electrical activity of the heart: the hERG channel mediates the repolarizing IKr current in the cardiac action potential, which helps coordinate the heart's beating.

KCNJ2 has the cardiovascular art-recognized meaning. KCNJ2 (Hs00265315_m1) encodes the Kᵢᵣ2.1 inward-rectifier potassium ion channel and is a lipid-gated ion channel.

KCNJ3 has the cardiovascular art-recognized meaning. Potassium inwardly-rectifying channel, subfamily J, member 3 (KCNJ3; HsM4334861_s1) encodes an integral membrane protein and inward-rectifier type potassium channel. The encoded protein, which has a greater tendency to allow potassium to flow into a cell rather than out of a cell, is controlled by G-proteins and plays an important role in regulating heartbeat.

KCNQ1 has the cardiovascular art-recognized meaning. KCNQ1 (Hs00923522_m1) encodes the I_{Ks} or slow delayed rectifier potassium channel. It plays an important role in cardiac action potential (AP) repolarization during β-adrenergic stimulation and participates in cardiac AP-rate-dependent adaptation. I_{Ks} mutations are implicated in the most common congenital long QT syndrome, Type 1.

MYL2 has the cardiovascular art-recognized meaning. MYL2 is the gene for myosin light chain 2v (MLC2v) and characteristic of cardiac ventricles. The protein is to form cardiac sarcomere, for the maintenance of ventricular contractility. MYL2 is a ventricular marker.

NPPA has the cardiovascular art-recognized meaning. NPPA encodes atrial natriuretic peptide, a hormone that is secreted from the cardiac atria.

NR2F2 has the cardiovascular art-recognized meaning. NR2F2 (nuclear receptor subfamily 2, group F, member 2; COUP-TFII; COUP transcription factor 2; Hs00818842_m1) encodes an atrial biomarker.

Optical mapping has the cardiovascular art-recognized meaning. Optical mapping is a technique for constructing ordered, genome-wide, high-resolution restriction maps from single, stained molecules of DNA, called “optical maps”. By mapping the location of restriction enzyme sites along the unknown DNA of an organism, the spectrum of resulting DNA fragments collectively serves as a unique “fingerprint” or “barcode” for that sequence. Later technologies use DNA melting, DNA competitive binding, or enzymatic labelling to create the optical mappings.

Retinoic acid (CAS Number 302-79-4) mediates the functions of vitamin A1 required for growth and development. Retinoic acid acts by binding to the retinoic acid receptor (RAR), which is bound to DNA as a heterodimer with the retinoid X receptor (RXR) in regions called retinoic acid response elements (RAREs). Binding of the all-trans-retinoic acid ligand to RAR alters the conformation of the RAR, which affects the binding of other proteins that either induce or repress transcription of a nearby gene (including Hox genes and several other target genes).

SCN5A has the cardiovascular art-recognized meaning. SCNSA (Hs00165693_ml) encodes an integral membrane protein (Naᵥ1.5.) and tetrodotoxin-resistant voltage-gated sodium channel (I_{Na}) subunit.

Three-dimensional (3D) microtissues has the cardiovascular art-recognized meaning.

Two-dimensional (2D) culture has the cardiovascular art-recognized meaning.

Ventricular cardiomyocyte (vCM) has the cardiovascular art-recognized meaning. Atrial and ventricular cardiomyocytes form the muscular walls of the heart (the myocardium). Atrial myocytes have a different ultrastructure compared to ventricular myocytes. They have differential gene expression patterns regarding, e.g., transcription factors, structural proteins, and ion channels. Ng, Wong, & Tsang, Differential gene expressions in atrial and ventricular myocytes: insights into the road of applying embryonic stem cell-derived cardiomyocytes for future therapies. Am. J. Physiol. Cell Physiol., 299(6), C1234-49 (December 2010). They also display distinct functions.

Unless otherwise defined herein, scientific and technical terms used with this application shall have the meanings commonly understood by persons having ordinary skill in the biomedical art. This invention is not limited to the particular methodology, protocols, and reagents, etc., described herein and as such can vary.

The disclosure described herein does not concern a process for cloning humans, processes for modifying the germ line genetic identity of humans, uses of human embryos for industrial or commercial purposes or processes for modifying the genetic identity of animals which are likely to cause them suffering with no substantial medical benefit to man or animal, and also animals resulting from such processes.

### Guidance From Materials and Methods

A person having ordinary skill in the art can use these materials and methods as guidance to predictable results when making and using the invention:

Cardiomyocyte differentiation. The inventors differentiated atrial and ventricular cardiomyocytes (aCMs/vCMs) from GCaMP6f-expressing human induced pluripotent stem cells (hiPSCs; WTC human male iPSCs, Gladstone Institutes, San Francisco, Calif., USA) using small molecule modulations of Wnt signaling, as described previously by Burridge et al., Nature Methods, 11, 855-860 (2014), with slight modifications. hiPSCs were cultured on vitronectin coated plates in Essential 8 Medium (E8 media; Thermo Fisher Scientific, Waltham, Calif., USA). Before starting differentiation, hiPSCs were singularized, seeded onto Matrigel-coated plates in E8 media with 5 μM ROCK Inhibitor (RI; Thermo Fisher Scientific, Waltham, Calif., USA) and cultured to 80% confluency, at which point the cells were treated with 4.5 μM CHIR 99021 (Tocris Bioscience, Minneapolis, Minn., USA), a glycogen synthase kinase 3 (GSK3) inhibitor, for 24±1 hours in chemically defined medium CDM3 basal medium (RPMI 1640 Thermo Fisher Scientific, Waltham, Mass., USA) supplemented with L-ascorbic acid and human serum albumin). Burridge et al., Nature Methods, 11, 855-860 (2014). At differentiation day 3, cells were treated with 5 μM IWP2 (Tocris Bioscience, Minneapolis, Minn., USA), a Wnt inhibitor, in a CDM3 media mixture containing half spent and half fresh media. Atrial-subtype differentiation was achieved by daily supplementation of 1 μM retinoic acid (RA) at days 3-6. Cyganek et al., JCI Insight, 3 (2018). Cells for ventricular specification did not receive retinoic acid treatment. The inventors differentiated both atrial and ventricular cardiomyocytes within each batch of differentiation for direct comparison of chamber-specific responses. They removed IWP2 at day 5 of differentiation. They replaced the CDM3 culture medium every other day. After the first signs of contraction between days 9-13, hiPSC-cardiomyocytes were maintained in Gibco RPMI Media 1640 (RPMI 1640) mammalian cell culture media with B27TM supplement (RPMI/B27) (Thermo Fisher Scientific, Waltham, Mass., USA). The inventors then harvested hiPSC-cardiomyocytes between days 13-15 with 0.25% trypsin in 0.5 mM EDTA and replated the cells to Matrigel-coated plates for metabolic-based lactate purification. Tohyama et al., Cell Stem Cell, 12, 127-137 (2013). At day 20, hiPSC-cardiomyocytes were fed with lactate media composed of four mM sodium L-lactate (MilliporeSigma, St. Louis, Mo., USA) in sodium pyruvate-free and glucose-free DM EM (Thermo Fisher Scientific, Waltham, Mass., USA; Catalog # 11966025) for four days, with media changes every other day. Purified hiPSC-cardiomyocytes were then cultured in RPMI/B27 and used for generating 3D microtissues between days 27-30.

A timeline summarizing the cardiac differentiation protocol is highlighted in FIG. 1A.

Human cardiac fibroblast maintenance. Human male cardiac fibroblasts (hCFs, MilliporeSigma, St. Louis, Mo., USA) were cultured in DMEM/F12 with 10% fetal bovine serum (FBS), 1% Pen/Strep, and four ng/mL basic fibroblast growth factor (Reprocell, Beltsville, Md., USA). Human cardiac fibroblasts between passage numbers P2-P4 were used to generate 3D microtissues to promote heterocellular crosstalk that aids tissue compaction and improves electrical conduction. Kim et al., PLoS One, 13, e0196714 (2018); Rupert, Kim, Choi, & Coulombe, Stem Cells Int., 2020, 9363809 (2020).

GCaMP evaluation of spontaneous beating rates. The inventors used GCaMP fluorescence to characterize the automaticity of atrial cardiomyocytes and ventricular cardiomyocytes in both 2D culture and 3D microtissues. The samples were imaged with an inverted fluorescent microscope (Olympus IX50) one-three days before and six days after 3D microtissue formation without electrical stimulation. Fluorescent images were acquired for fifteen seconds for an accurate estimate of signal periodicity across multiple beats. Background fluorescence was removed and changes in fluorescence signal intensity corresponding to the intracellular calcium transients (CaT) of the beating cardiomyocytes were plotted using a custom MATLAB script (FIG. 1B). An automated peak-detection algorithm was implemented to quantify the period between beats and the width of GCaMP signal, and their averages used to estimate the frequency of spontaneous activity.

3D microtissue generation. Sterile 2% (wt/vol) agarose in PBS were pipetted into 35-microwell molds with hemispherical bottoms (FIG. 1C; 3D Petri Dish®, MicroTissues Inc., see MilliporeSigma, St. Louis, Mo., USA). After casted, hydrogels equilibrated in RPMI/B27 media with 1% Pen/Strep overnight in an incubator. Lactate-purified hiPSC-cardiomyocytes were then harvested, singularized, and suspended in RPMI/B27 media with 10% FBS and 1% Pen/Strep, collecting a subset of the cells for flow cytometry analysis of cTnT and MLC2v expression. The inventors added 5% human cardiac fibroblasts of the total number of hiPSC-cardiomyocytes to the cell suspension and pipetted the cell mixture to the center of the hydrogel at a density of 500-700,000 cells/hydrogel, producing thirty-five individual microtissues consisting of 15-25,000 cells/microtissue. Cells were allowed to settle into the cylindrical recesses for 30 minutes before adding media supplemented with 5 μM ROCK Inhibitor. Culture medium was changed one day post-seeding and replaced every other day. Self-assembling, spheroidal, and scaffold-free 3D microtissues were electrically field stimulated for six-eight days with a 1 Hz, 10.0 V, and 4.0 milliseconds duration bipolar pulse train (C-Pace EP, IonOptix, Westwood, Mass., USA) to precondition the microtissues before optical mapping. Radisic et al., Proc. Natl. Acad. Sci., U.S.A., 101, 18129-18134 (2004). The 3D microtissues beat synchronously with 1 Hz stimulation within two days, and tissue compaction were observed within three days post-seeding.

Flow cytometry. Samples for flow cytometry were fixed in 4% paraformaldehyde for ten minutes in the dark at room temperature and permeabilized with 0.75% saponin in PBS. Cells were stained with 1:100 mouse monoclonal IgG1 cTnT (Invitrogen; Catalog#: MA5-12960; Clone 13-11) and 1:10 monoclonal IgG1 myosin light chain 2v (MLC2v conjugated to APC; Miltenyi Biotec, San Diego, Calif., USA; Catalog#: 130-106-134) for 1 hour. Secondary staining was performed with 1:200 goat anti-mouse IgG PE (Jackson; Catalog#: 115-116-072) for one hour. cTnT⁺ cells were used to determine cardiomyocyte purity, and MLC2v^{+/-} cells were used to distinguish between ventricular and atrial subtypes, respectively. Samples were run on a BD FACSAriaTM Illu Flow Cytometer (BD Biosciences, San Jose, Calif.), and data were analyzed with FlowJo (BD Biosciences, San Jose, Calif.).

Optical mapping of cardiac action potential. The inventors transferred hydrogels containing microtissues to a Petri dish on a temperature-controlled chamber (Dual Automatic Temperature Controller TC-344B, Warner Instrument, Hamden, Conn., USA) to maintain ambient temperatures of 35±1° C. throughout imaging. Microtissues were gently perfused in a solution containing (in mM) 140 NaCl, 5.1 KCl, 1 MgCl₂, 1 CaCL₂, 0.33 NaH₂PO₄, 5 HEPES, and 7.5 glucose warmed with an inline heater. Microtissues were allowed to equilibrate in the perfusion solution for thirty minutes and subsequently labeled with a voltage-sensitive dye (5 μM di-4-ANEPPS) for five minutes to enable membrane potential (Vₘ) recordings of action potential. Excess residual dyes were washed out thoroughly before data collection. An active-pixel sensor (CMOS sensor) camera acquired fluorescence images at 1000 frames-per-second, and action potential traces were reconstructed from fluorescence intensity data. Semi-automated analyses of action potential parameters were conducted using an in-house analysis software and included rise time, APD₃₀, APD₅₀, APD₈₀, APD to maximum repolarization rate (APD_{MXR}), and APD triangulation (APDₜᵣᵢ).

An in-depth description on the post-processing of optical mapping data for action potential analysis is detailed in a previous work by the inventors. Kofron et al., Sci. Reports, 11(1),10228 (2021). This description is summarized in FIG. 1D.

Screening of arrhythmogenic compounds. The inventors studied the effects of 4-aminopyridine (4-AP), an Iₜₒ and I_{Kur}blocker, and ivabradine, an I_{f} blocker, on the action potential properties of 3D cardiac microtissues. Three molds of microtissues (two atrial cardiomyocytes and one ventricular cardiomyocytes) were used to test the compound 4-aminopyridine under 1 Hz electrical stimulation with a platinum field electrode (Myopacer EP field stimulator, IonOptix, Westwood, Mass., USA). Baseline action potential recordings were acquired before three increasing dosages of 4-aminopyridine (1 μM, 3 μM, and 100 μM) were introduced into the perfusion solution. At each dose, the microtissues were allowed to respond to the drug for five-ten minutes before action potential recordings were acquired. At least ten seconds of data were acquired per microtissue at each dose. Instead, three molds of atrial microtissues were used to test the compound ivabradine without electrical stimulation to investigate how action potential properties and spontaneous activity were altered in response to the drug. The inventors did not test ivabradine on ventricular microtissues because the microtissues do not exhibit spontaneous action potentials. Three different dosages of ivabradine (1 μM, 2 μM, and 3 μM) were tested independently, instead of in succession, because the loss of spontaneous activity following drug treatment could not be recovered.

Human cardiac fibroblast culture. Patent publication WO 2020/23243 (Brown University) discloses the following method, which persons of ordinary skill in the art may want to use in the practice of this invention. Human cardiac fibroblasts (hCFs, from PromoCell or Sigma-Aldrich) were maintained and passaged in DMEM/F12 supplemented with 10% FBS, 1% P/S, and four ng/ml bFGF. Cells were passaged upon reaching near confluency in versene with 0.05% trypsin (ThermoFisher). For some studies of hCF phenotype, coverslips were coated with polyacrylamide gels at 10% acrylamide and 0.1% bis-acrylamide for a stiffness of approximately 12 kPa. Gels were functionalized with 0.2 mg/mL human Fibronectin (Sigma Aldrich) and seeded with hCFs for at least 72 hours. Human cardiac fibroblasts were incorporated into cardiac microtissues at cell passages P2-P4 or in engineered macro-tissues at cell passages P4 (young, healthy, quiescent) or P9 (aged, activated, disease-like, myofibroblast). Tissues containing hCFs demonstrate higher quality, as assessed by consistent formation, smoother edges, and improved electromechanical function (quantified by excitability, action potential waveform shape, and action potential duration) this are essential for cardiotoxicity evaluation.

Fabrication of microtissue mold hydrogels and 3D microtissue culture. Patent publication WO 2020/23243 (Brown University) discloses the following method, which persons of ordinary skill in the art may want to use in the practice of this invention. Scaffold-free three-dimensional microtissues (spheroid in shape, also called spheroids and/or organoids) are generated using non-adhesive agarose gels with cylindrical microwells with hemispherical bottoms to guide self-assembly. See FIG. 1(B). Sterilized 2% (wt/vol) agarose is pipetted into molds designed for 24-well plates with 800-μm-diameter rounded pegs (Microtissues, Providence, R.I., USA). After being cooled to room temperature (−5 minutes), the agarose gels are separated from the molds and transferred to single wells of 24-well plates. For equilibration, 1 mL medium is added to each well. Hydrogels are equilibrated at least one hour or overnight at 37° C. in a humidified incubator with 5% CO2. Molds are transferred to 6-well plates for electrical stimulation, and hiPSC-CM or hiPSC-CM LP with or without additional human cardiac fibroblasts (5-15%) in suspension are added to the center of the hydrogel seeding chamber (100-900K cells/mold in 35 recesses, depending on output being assessed; typically, 600-800K for optical mapping) and allowed to settle into the recesses for 30 minutes. Medium is then added to each well (5 ml), and cells are cultured for 6-8 days with electrical field stimulation with a 1 Hz, 10.0 V, 4.0 milliseconds duration bipolar pulse train for the full three-dimensional culture period (C-Pace EP, IonOptix).

Image acquisition and processing. Patent publication WO 2020/23243 (Brown University) discloses the following method, which persons of ordinary skill in the art may want to use in the practice of this invention. Phase-contrast images of cells and microtissues were captured with a Nikon TE2000-U and a black and white/color digital camera (MicroVideo Instruments, Avon, Mass., USA) and acquired and analyzed with NIS Elements software.

Microtissue size analysis. Patent publication WO 2020/23243 (Brown University) discloses the following method, which persons of ordinary skill in the art may want to use in the practice of this invention. Stitched 4× phase-contrast images of whole 35-well microtissue hydrogels were acquired and analyzed. Image thresholding and particle size analysis was used in NIS Elements to determine the top view cross-sectional area of individual microtissues across each mold.

3D tissue sections and immunohistochemistry. Patent publication WO 2020/23243 (Brown University) discloses the following method, which persons of ordinary skill in the art may want to use in the practice of this invention. The inventors fixed microtissues in 35-well hydrogels using 4% (vol/vol) paraformaldehyde (Electron Microscopy Sciences, Hatfield, Pa., USA) and 8% (wt/vol) sucrose in phosphate-buffered saline (PBS) overnight at room temperature. Molds were then rinsed twice with phosphate-buffered saline and equilibrated, as indicated by their sinking, usually over twelve hours, with 15% and then 30% (wt/vol) sucrose in phosphate-buffered saline. Whole agarose gels containing microtissues were removed from sucrose, blotted dry, and embedded in Tissue-Tek CRYO-OCT Compound (Ted Pella, Redding, Calif., USA). Blocks were stored at −80° C., sectioned on a Leica CM3050 cryostat microtome (Leica Biosystems, Buffalo Grove, Ill., USA) into 10 pm-thick sections, and placed on Superfrost Plus slides. After being air dried for fifteen minutes, sections were postfixed in 4% paraformaldehyde in phosphate-buffered saline. For immunofluorescent staining at room temperature, frozen sections were rinsed three times for five minutes with 1× phosphate-buffered saline wash buffer. Non-specific binding was blocked with 1.5% goat serum for one hour, followed by one-hour incubations in primary and secondary antibodies diluted in 1.5% goat serum. Primary antibodies were directed against cardiac troponin I (cTnl, 1:100, Abcam ab47003) and vimentin (1:100, Sigma-Aldrich (St. Louis, Mo., USA) V6630), and secondary antibodies were conjugated to Alexa Fluor 488 or Alexa Fluor 594 (1:200, Invitrogen). Coverslips were mounted with Vectashield mounting medium with DAPI. Images were taken with an Olympus FV3000 Confocal Microscope and processed using ImageJ.

Optical signals. Patent publication WO 2020/23243 (Brown University) discloses the following method, which persons of ordinary skill in the art may want to use in the practice of this invention. The optical signals of cardiomyocyte excitation are simple and compatible with rapid analysis. The source of the optical signal varies. Fluorescing dyes can detect voltage and calcium. These two signals have physiological relevance, as voltage is the measure of the action potential, and the action potential triggers intracellular calcium to rise, so the intracellular calcium concentration gives a measure of the calcium transient (CaT). Alternative dyes with longer wavelength are being developed, which could be used in the invention, and some genetically encoded voltage- and calcium-responsive fluorescent proteins are available if human induced pluripotent stem cell lines are engineered to express these reporters, which itself involves an investment of labor and resources. Following the action potential and calcium transient in cardiomyocytes is a physical muscle contraction, and this signal can be detected optically through movement of the cells, tissue, or posts where the tissue is attached. The “Biowire” platform now being commercially developed by Tara Biosciences, uses fluorescent wires through the ends of three-dimensional tissues so the contraction can be extracted through optical detection of wire deflection. However, a contractile signal for arrhythmia detection is two steps removed from the source of the signal (which is the action potential) and like in the game of “telephone” the smoothing and distortion of the signal can complicate the data interpretation for arrhythmias. For all these signals, the spatial and temporal resolution of these signals varies based on the equipment used, and this resolution impacts the precision of the measurements and their interpretation. Because arrhythmias are triggered primarily by changes in the action potential and less often by changes in the calcium transient or contraction, the precision of the metrics are of paramount importance for assessing arrhythmic cardiotoxicity.

Optical mapping and automated action potential duration analysis. Patent publication WO 2020/23243 (Brown University) discloses the following method, which persons of ordinary skill in the art may want to use in the practice of this invention. The inventors used an Olympus MVX10 microscope to image 1.2×1.2−mm2 regions. Microtissues were loaded with voltage-sensitive di-4-ANEPPS (5 μM for ten minutes at 35° C.) for measurements of membrane potential (Vm). The inventors acquired and analyzed fluorescence images at 979 frames/s using a Photometrics Evolve+128 EMCCD camera (2×2 binning to 64×64 pixels, 18.7×18.7−μm2 resolution, 1.2×1.2−mm2 field of view) and an Olympus MXV10 macroview optical system. Fluorescence images were filtered using nonlinear bilateral filter (spatial filter: 5×5 window, temporal filter: 21-point window) to preserve action potential upstrokes from blurring. Typically, four microtissues were recorded simultaneously/scan at this magnification. A single microtissue is typically covered by ˜60 pixels at this magnification. The pixels with action potentials were identified from Fast Fourier transformation (FFT) of fluorescence signals. After appropriate thresholding and image segmentation, the region of each microtissue was grouped and the fluorescence signals from the pixels in the same microtissue were average and used for action potential analysis.

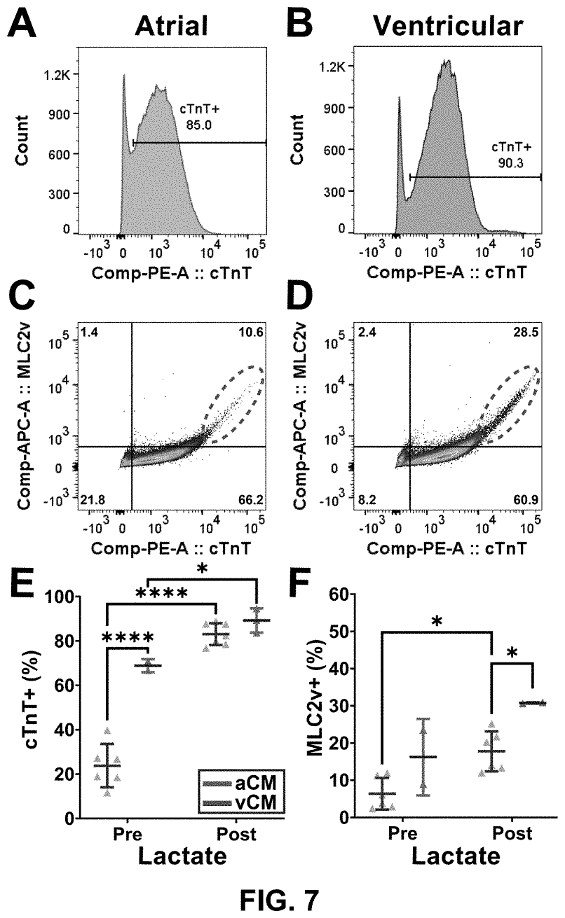

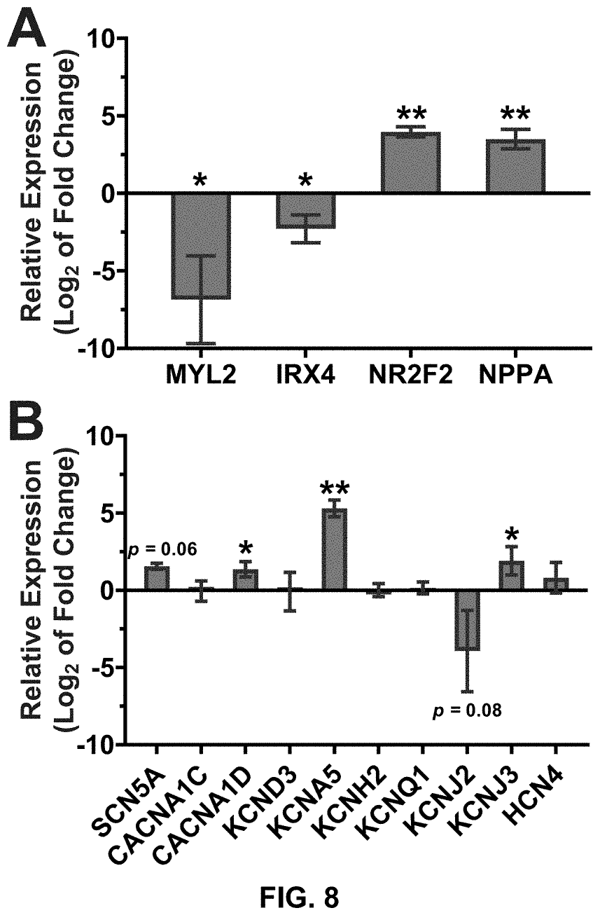

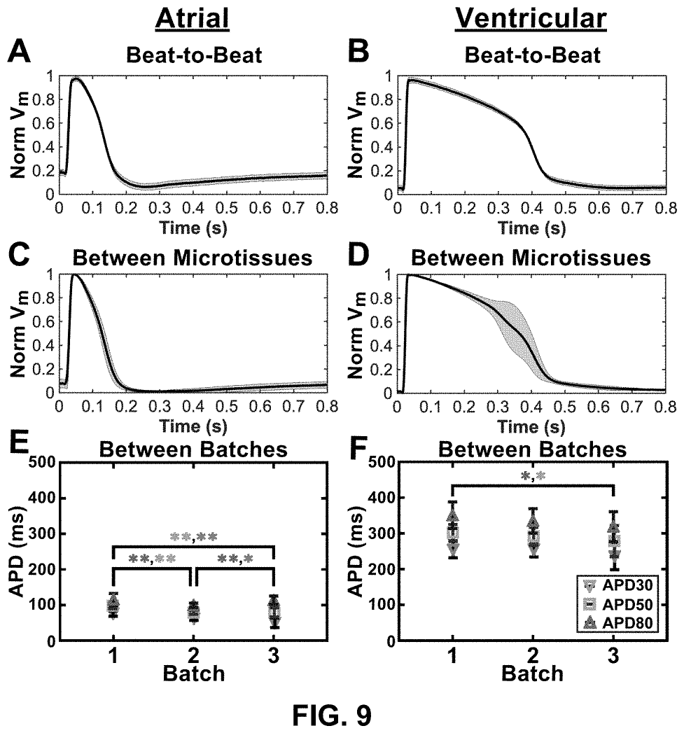

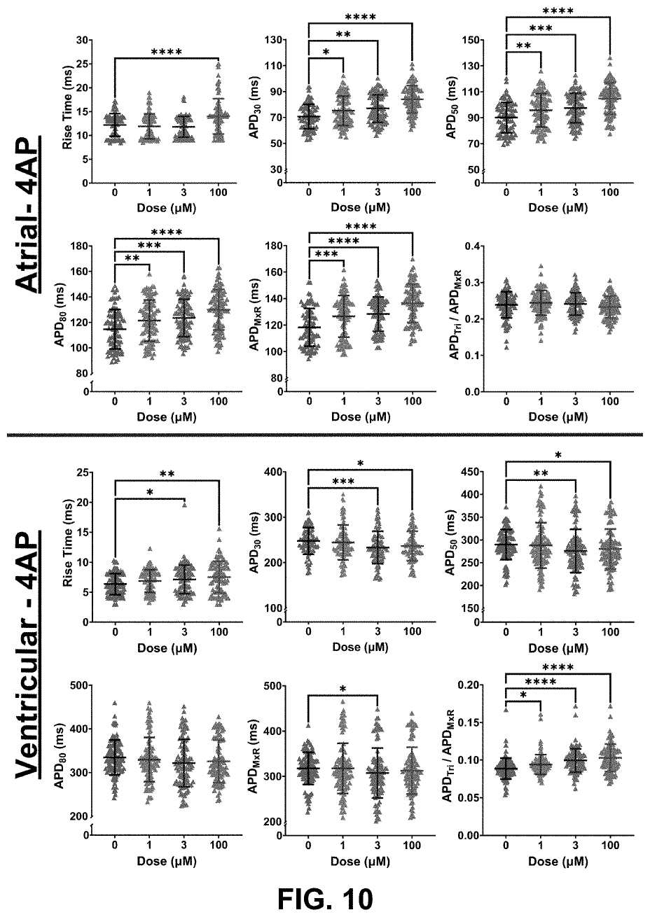

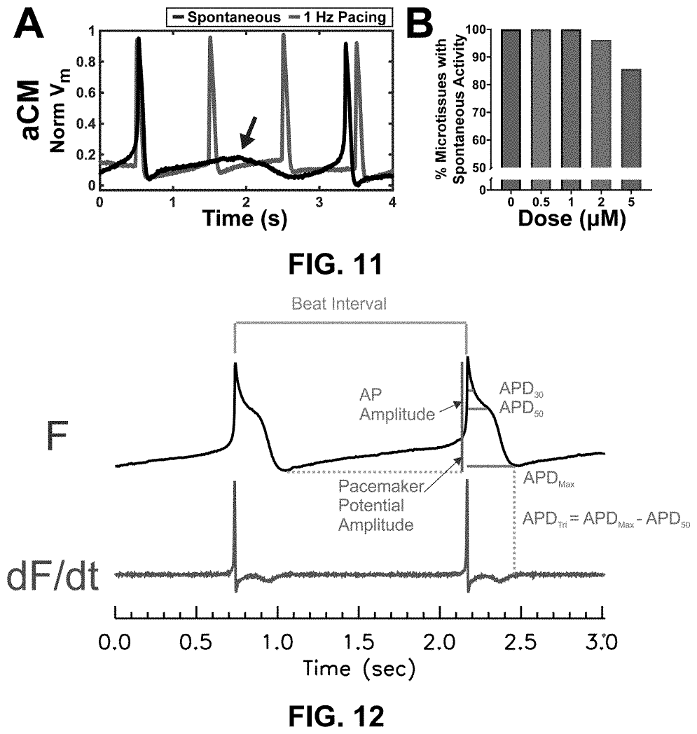

Validation and screening of toxicants for arrhythmogenic risk. Patent publication WO 2020/23243 (Brown University) discloses the following method, which persons of ordinary skill in the art may want to use in the practice of this invention. Microtissues were acutely exposed to increasing concentrations of E4031 (a high-risk HERG channel blocker; 0-2 μM), ranolazine (a low-risk sodium channel and HERG blocker, 1-100 μM), and bisphenol-A (at 1-1000 nM) with 20-minute incubation periods followed by approximately three-minute imaging periods. Concentrations are selected to span human exposure levels or blood serum levels and quantify dose-dependent changes over a wide range (with a goal of more than 10,000× change in concentration and at least 4-6 doses). A single mold of microtissues is imaged for approximately 1 hour to assure quality recordings without signal degradation due to tissue degeneration, enabling measurement under control conditions (zero compound) and three doses. Small changes are quantified by AP metrics and discrimination between compounds targeting HERG channel (E4031 and ranolazine) is demonstrated (see FIG. 7 and FIG. 8).

Quantitative RT-PCR. Patent publication WO 2020/23243 (Brown University) discloses the following method, which persons of ordinary skill in the art may want to use in the practice of this invention. Messenger RNA was extracted from cells (CMs or hCFs) and engineered tissues using the RNeasy Mini Kit and mRNA concentration was measured with a NanoDrop 1000 Spectrophotometer. The cDNA was synthesized from a normalized mass of mRNA for cells and tissues separately using the SuperScript III First-Strand Synthesis System. Complimentary DNA (cDNA) samples were combined with custom primers and SYBR Master Mix, and quantitative real-time PCR was run on an Applied Biosystems® 7900 fast real-time system. HPRT was an internal control for normalization and relative expression was calculated using the 2\hat{ }(−ΔΔCt) method. Livak & Schmittgen, Methods, 25(4), 402-408 (2001).

Macro-tissue mold and tissue formation. Patent publication WO 2020/23243 (Brown University) discloses the following method, which persons of ordinary skill in the art may want to use in the practice of this invention. Molds for larger macro-sized engineered tissues with mm to cm dimensions and tissues formed in them are created as previously described by Munarin et al. (2017) and Kaiser et al. (2019). In brief, custom acrylic molds were fabricated by laser etching/cutting using a 100 W CO2 laser and polydimethylsiloxane (PDMS) was poured into acrylic negatives and cured at 60° C. PDMS molds were sterilized by autoclaving. Tissues are form by combining 1×106 hiPSC-CMs and 0-15% hCFs with 1.6-3.2 mg/mL rat tail collagen-1 at a 50%/50% vol/vol ratio for a final concentration of approximately 16×106 hiPSC-CMs/mL and 0.8, 1.25, or 1.6 mg collagen/mL. Cell-collagen solution was pipetted into PDMS molds, maintained in RPMI/B27, and stimulated with a four millisecond biphasic pulse at 1 Hz and 5 V/cm for the duration of culture.

Mechanical testing. Patent publication WO 2020/23243 (Brown University) discloses the following method, which persons of ordinary skill in the art may want to use in the practice of this invention. Mechanical measurements were performed after one or two weeks of culture as previously described. Engineered tissues were cut in half and their passive and active mechanical properties were measured with an ASI 1600A system. Aurora Scientific, Ontario, Canada. Strips were mounted on hooks attached to a 5 mN force transducer and high-speed motor arm, bathed in Tyrode's solution with 5 mM glucose and 1.8 mM CaCl2 at 30-34° C., and electrically field stimulated with platinum electrodes. Tissues were stretched from their initial length, Lo (determined as just above slack length), by 5% steps to 130% _{L0}. At the final length, tissues were paced with increasing frequency, and the fastest pacing they followed was recorded as the maximum capture rate (MCR).

Calculations were made from the data recorded during mechanical testing to obtain these values: Patent publication WO 2020/23243 (Brown University) discloses the following method, which persons of ordinary skill in the art may want to use in the practice of this invention. Active stress, σa, was calculated by averaging the active twitch force of ten contractions and normalizing by the cross-sectional area (CSA). The was calculated under the assumptions that tissue height was half the width and cross-sectional shape was an ellipse. Fold change was calculated from the ratio of the maximum active stress at 130% L₀ to active stress at the initial length L₀. Passive stress, σp, was calculated by normalizing the passive (baseline) force produced at each step by the cross-sectional area, and tissue stiffness (Young's modulus) was calculated as the slope of the line of best fit of passive stress versus strain at 5-30% strain.

Computational modeling. The inventors relied on computational modeling to recapitulate the effects of I_{Kur} inhibition by 4-aminopyridine on the action potential properties of hiPSC- atrial cardiomyocytes. the model was based on the 2015 hiPSC-cardiomyocytes action potential model equations established by Paci et al., Ann. Biomed. Eng., 41, 2334-2348 (2013) and Br. J. Pharmacol., 172, 5147-5160 (2015). The Paci model was derived from patch-clamp I-V curves and action potential data of atrial and ventricular-like immature hiPSC-cardiomyocytes, and included the major currents (I_{Na}, Iₜₒ, I_{CaL}, I_{K1}, I_{Kr}, I_{Ks}, and I_{f}) pump/exchanger currents (I_{NaK}, I_{pCa}, and I_{NaCa}), Ca²⁺ dynamics and buffering in the sarcoplasmic reticulum, and background currents. See, Ma et al., Am. J Physiol. Heart Circ. Physiol., 301, H2006-2017 (2011). However, the Paci model did not include the atrial-specific ultrarapid voltage gated repolarizing potassium current I_{Kur}, which the inventors added based on the action potential model equations published by Maleckar et al., Am. J. Physiol. Heart Circ. Physiol., 297, H1398-1410 (2009), for adult human atrial CMs.

The following set of equations for I_{Kur} were incorporated into the Paci model:

I_{Kur}=g_{Kur}×aᵤᵣ×iᵤᵣ(V−E_{K}) Equation 1

daᵤᵣ/dt=(aᵤᵣ∞−aᵤᵣ)/τₐᵤᵣ Equation 2

diᵤᵣ/dt=(iᵤᵣ, ∞−iᵤᵣ/τᵢᵤᵣ Equation 3

aᵤᵣ,∞=1.0/[1.0+e^{-(V+6)/8.6}] Equation 4

iᵤᵣ, ∞=1.0/[1.0+e^{(V+7.5)/10.0}] Equation 5

τₐᵤᵣ=0.009/[1.0+e^{(V+5.0)/12.0}]+0.0005 Equation 6

τᵢᵤᵣ=0.59/[1.0+e^{(V+60.0)/10.0}]+3.05 Equation 7

where g_{Kur} is the maximum conductance, aᵤᵣ is the activation gating variable, iᵤᵣ is the inactivation gating variable, and τ_{a/iur} is the activation/inactivation time constant for I_{Kur}. V corresponds to the membrane potential and E_{K} corresponds to the Nernst potential for K⁺. To appropriately scale I_{Kur} for immature hiPSC-cardiomyocytes, the inventors tuned the maximum conductance of the ultrarapid potassium channel, together with the other major ion channels, by minimizing the sum of square residuals between the modeled and experimentally measured averaged action potential waveforms, without altering the activation/inactivation kinetics of the ion channels. All other parameters remained unchanged from the original Paci model. TABLE 1 summarizes the different maximum conductance parameters for the major ion channels that were modified from the Paci model to best match the experimental action potential data.

TABLE 1. Major ion channel conductances, summarizes the major ion channel conductance values that were modified from the 2015 Paci model to match action potential waveforms obtained experimentally from the atrial and ventricular 3D microtissues.

TABLE 1
Major ion channel conductances

| Max, | | | | |
|---|---|---|---|---|
| conductance | Atrial | | Ventricular | |
| g_{Na} | 1.9939e3 | (S/F) | 2.3262e3 | (S/F) |
| g_{CaL} | 5.1814e−5 | (m³/(F × s)) | 8.6357e−5 | (m³/(F × s)) |
| gₜₒ | 59.8077 | (S/F) | 14.9519 | (S/F) |
| g_{Kur} | 0.01875 | nS | N/A | |
| g_{Kr} | 31.360 | (S/F) | 43.3067 | (S/F) |
| g_{Ks} | 2.041 | (S/F) | 3.0615 | (S/F) |
| g_{K1} | 19.1925 | (S/F) | 36.5940 | (S/F) |
| gᵣ | 75.2578 | (S/F) | 21.0722 | (S/F) |

Atrial differentiation and 3D cardiac microtissue generation. A protocol is shown in EXAMPLE 1 below.

Analysis routine. Two additional atrial specific metrics are added to a standard protocol: (1) pacemaker potential amplitude and slope, (2) APDₘₐₓ that measures APD through detecting the maximum hyperpolarization point. See FIG. 12.

Pacemaker potential amplitude is measured by detecting the threshold potential, a start point when the action potential upstroke starts (FIG. 12, red line). The raw trace is first normalized with ΔF/F₀=(F−F₀)/F₀ where F is fluorescence value and F₀ is the baseline fluorescence value. Then, the 2^{nd} derivative of ΔF/F₀ is calculated. The threshold potential is automatically detected from the maximum of 2^{nd} derivative of action potential trace which coincidentally occurs at the threshold potential. The slope of pacemaker potential (the pacemaker amplitude divided by the time between the repolarization time point of the previous action potential and the following action potential takeoff time point) is a measure of risks for proarrhythmic automaticity.

The maximum hyperpolarization point is automatically detected by choosing the critical point where membrane potential changes from repolarization to depolarization. This is a local minimum point where the 1^{st} derivative of ΔF/F₀ changes from negative to positive. See FIG. 12. This algorithm measures maximum length of APD compared to APD₇₅ or APD₉₀that can be affected by baseline drift.

Data acquisition steps. These data acquisition steps are outlined in the methods section of the manuscript under sub-headers “Optical Mapping of Cardiac Action Potential” And “Screening of Arrhythmogenic Compounds (Soepriatna, et al. Cell. Mol. Bioeng., 14, 441-457 (2021), briefly summarized below:

Transfer the microtissue mold into the temperature regulated chamber in the optical mapping apparatus

Add voltage sensitive dye to our microtissues (˜one minute).

Rinse the dye off with the perfusion solution

Perfuse the perfusion solution using syringe pump at three ml/min for ten minutes until the microtissues have a stable baseline measurement

Test drug at low concentration by perfusion drug solution for five-ten minutes.

Record action potential

Repeat last two steps above with increasing drug dose.

Flow cytometry is a standard and widely known cytometry method. Many flow cytometry protocols are available.

Many cell culture media are commercially available, including E8 (for use with seed hiPSCs), RPMI/B27 (for use at 9-20 days), lactate media (for use at 20-24 days), RPMI/B27 (for use at 24 to 27 or 30 days), and RPMI/B27+10% FBS+1% Pen-Strep (for use at 27 or 30 days to 33 or 38 days).

CDM3 media (for use 0-9 days), is made based on a publication by Burridge et al. Nat Methods, 2014 (see new word document on atrial differentiation and tissue formation).

Statistical analyses. All data were reported as mean±standard deviation and tested for normality with the Shapiro-Wilk test. A Student's two tailed unpaired t-test was used to study the effects of cardiac subtype on the different action potential metrics, calcium transients, and automaticity. For compound testing experiments, a one-way analysis of variance with Dunnett's multiple comparison to baseline controls was performed for all AP metrics with normal distribution. Statistical analyses of non-normal data were performed with the nonparametric Kruskal-Wallis test. All statistical tests were conducted in GraphPad Prism version 8.1.1 (GraphPad Software) with p<0.05 representing statistical significance.

The following EXAMPLES are provided to illustrate the invention and shall not limit the scope of the invention.

### EXAMPLE 1

### Atrial Differentiation and 3D Cardiac Microtissue Generation Protocol

### Protocol Adapted From:

(1) GiWi Protocol: Lian et al., Nat Proc, (2013).

(2) Atrial Differentiation: Cyganek et al., JCI Insight (2018.

(3) 3D Cardiac Microtissues: Soepriatna et al., Cell Mol Bioeng, (2021).

(4) Kofron et al., Science Reports (2021).

### Reagents/Materials: General Reagent:

DPBS, No Calcium, No Magnesium (ThermoFisher, Catalog#: 14190144)

Fetal Bovine Serum (FBS) (ThermoFisher, Catalog#: 16000044)

Penicillin-Streptomycin (Pen-Strep) (Sigma, Catalog#: P0781)

### Reagents/materials: For Plate Coating:

Vitronectin (VTN-N; 500 μg/mL) (ThermoFisher, Catalog#: A14700)

Coat 10 cm tissue culture plate with 5 μg/mL VTN-N (1:100 dilution in DPBS).

Coat for one hour at room temperature or overnight in humidity chamber at 4° C.

Matrigel™, Growth Factor Reduced (Fisher, Catalog#: CB356238)

Coat 24/12/6 tissue culture well-plate with 1× Matrigel™ (diluted in DMEM, high glucose, pyruvate [ThermoFisher, Catalog#: 11995073]).

Coat for 1 hour at 37° C. or overnight in humidity chamber at 4° C. Reagents/Materials: Culture Medium:

Essential 8™ Medium+Supplement (ThermoFisher, Catalog#: A1517001)

RPMI 1640 Medium (ThermoFisher, Catalog#: 11875093)

Cardiac differentiation media with three components (CDM3)

RPMI 1640 Medium supplemented with 213 pg/mL L-ascorbic acid and 500 g/mL human serum albumin (Adapted from Burridge et al, Nat Methods, 2014)

B27 Supplement (ThermoFisher, Catalog#: 17504044)

DMEM, No glucose, No Sodium Pyruvate (ThermoFisher, Catalog#: 11966025)

### Reagents/Materials: For Differentiation & Purification:

Chiron (CHIR 99021) (ToCris, Catalog#: 4423)

Inhibitor of Wnt Protein 2 (IWP2) (ToCris, Catalog#: 3533)

Retinoic Acid (Sigma, Catalog#: R2625)

Sodium L-Lactate (Sigma, Catalog#: L7022)

### Reagents/Materials: For Harvesting Cells:

Versene

Mix together 1L DPBS, 1mL of 0.5M EDTA, and 0.2 g of Dextrose

pH to 7.2-7.4 then sterile filter

TrypLE 10× Select (ThermoFisher, Catalog#: A1217702)

Trypsin (ThermoFisher, Catalog#: 27250018)

Y-27632 dihydrochloride (Rock Inhibitor, RI) (ToCris, Catalog#: 1254)

DNase I (Sigma, Catalog#: 10104159001)

Reagents/materials: For microtissue generation:

UltraPure™ Agarose (ThermoFisher, Catalog#: 16500100)

35-microwell molds (3D Petri Dish®) (MicroTissues Inc., 35 Large Spheroids)

Electrical pacing system with stimulator lid that is compatible with 24/12/6 well-plate (e.g., C-Pace EP by lonOptix with a 6 well-plate stimulator lid).

### Protocol

**Ensure that all media and plates are at room temperature or 37° C. before doing cell work.

Step 1: hiPSC Maintenance on VTN-N Coated 10 cm Plates

Feed with 10 mL complete Essential ^{8TM} Medium (E8) daily.

When plate reaches 80-90% confluency, passage hiPSC to VTN-N coated plates as described in steps 2.1-2.9.

Step 2: Cell Seeding for hiPSC Maintenance and Cardiac Differentiation

Aspirate spent media and rinse a plate of hiPSCs with 5 mL DPBS.

Aspirate DPBS and add 5 mL Versene.

Incubate plate at 37° C. for 4-5 minutes or until cells appear to dislodge from the bottom of the plate, as observed under a microscope. Tap plate gently to fully lift off cells.

Add 5 mL E8 to plate to neutralize Versene. Wash and collect cells in 50 mL conical.

Wash plate with another 5 mL E8 to harvest any remaining cells. Add to 50 mL conical.

Spin down hiPSCs at 200 g for four minutes.

While cells spin down, aspirate VTN-N from a new 10 cm plate and add 9 mL E8. Swirl to cover the entire plate.

Remove hiPSCs from centrifuge, aspirate supernatant, and resuspend pellet in 10 mL E8, triturating 3-5 times to break up pellet into small clusters.

Transfer 1 mL of suspended cells to VTN-N plate (1:10 split) and rock plate gently to evenly distribute cells. Incubate at 37° C.

Take 10 μL aliquot of leftover suspended cells and dilute 1:1 in Trypan blue (10 μL) in an Eppendorf tube. Count hiPSCs with hemacytometer.

Transfer suspended cells into a new 50 mL conical and dilute with E8 to reach desired seeding density in a total volume of 24 mL. (See TABLE 2 below for recommended seeding densities.)

Triturate hiPSC suspension into single cells. Add 5 μM RI.

Aspirate Matrigel from plate and divide cell suspension evenly into each well. Rock plate gently to evenly distribute cells. Denote seeding day as Day minus 1 of cardiac differentiation.

TABLE 2
Recommended seeding density and volume of
media per well

| Plate Format | Cell number | Volume/well |
|---|---|---|
| 24 well plate | 0.07-0.10 × 10⁶ cells/well | 1 mL/well |
| 12 well plate | 0.15-0.20 × 10⁶ cells/well | 2 mL/well |
| 6 well plate | 0.25-0.50 × 10⁶ cells/well | 4 mL/well |

Step 3: GiWi Monolayer Cardiac Directed Differentiation of Human Induced Pluripotent Stem Cells (hiPSCs)—Ventricular Subtype (black) and Atrial Subtype (red)

Day 0: hiPSC at ˜80% confluency. Check plate under a microscope and confirm that plates are ∞80-90% confluent. Then, remove E8, rinse wells with DPBS, and add CDM3 with 3.5-6 μM Chiron.

Note: 80-90% confluency is important for robust differentiation.

Note: Optimal Chiron concentration may vary based on cell line.

Day 1 (24±1 hours later): Remove CDM3 containing Chiron, rinse wells with DPBS, and feed with CDM3.

Day 3 (72±3 hours after Chiron): Feed with a media mixture containing half spent and half fresh CDM3 media+5 μM IWP2.

In a 50 mL conical, add 12 mL fresh CDM3+10 μM IWP2.

Collect half of the spent media volume from each well into conical containing fresh CDM3-IWP2 mixture.

Aspirate off remaining volume from wells.

Feed cells with CDM3 mixture of IWP2.

(For atrial subtype) Add 0.5-1 μM of retinoic acid into each well dedicated for atrial differentiation (OR if performing only atrial differentiation with no ventricular controls, mix 0.5-1 μM of retinoic acid into CDM3 mixture).

(For atrial subtype) Day 4: Add 0.5-1 μM of retinoic acid into each well dedicated for atrial differentiation only. No need to feed with fresh CDM3 media.

Day 5: Aspirate spent media, rinse wells with DPBS, and feed with fresh CDM3.

(For atrial subtype) Add 0.5-1 μM of retinoic acid into each well dedicated for atrial differentiation only.

Robust web formation should have occurred by this timepoint.

On Day 5, the inventors observed and took photographs of differentiation showing robust web formation.

(For atrial subtype) Day 6: Add 0.5-1 μM of retinoic acid into each well dedicated for atrial differentiation only. No need to feed with fresh CDM3 media.

Day 7: Aspirate spent media, rinse wells with DPBS, and feed with fresh CDM3.

Day 9: Aspirate spent media and add RPMI 1640 supplemented with B27 (RPMI/B27) even if cells have not begun to beat.

Day 11: Aspirate spent media and add RPMI/B27 even if cells have not begun to beat.

Note: Although ventricular cardiomyocytes (vCMs) should have begun to beat by this day, atrial cardiomyocytes (aCMs) may not have (aCMs tend to beat a few days after ventricular controls).

Day 13: Aspirate spent media and add RPMI/B27.

Note: All cells should be beating by this day with extensive web-like structures. aCMs will visibly exhibit shorter contractions and greater chance of developing spiral/re-entry arrhythmia than vCMs.

On Day 13, the inventors observed and took photographs of atrial differentiation showing extensive webbing.

### Step 4: Harvesting and Replating Cardiomyocytes (Day 13-15)

### Preparation:

Add 10 μM RI into each well and incubate plate at 37° C. for at least one hour.

Prepare and warm stop solution.

RPMI/B27+200 U/mL DNase I+10% FBS.

Prepare and warm replate solution.

RPMI/B27+10% FBS+5 μM RI+1% Pen-Strep.

Prepare either (1) TrypLE 10× Select or (2) warm 0.25% Trypsin in Versene (37° C.).

### Harvest:

Wash each well with DPBS.

Wash each well with Versene.

Add dissociation reagent. For TrypLE 10× Select:

Incubate cells at 37° C. Check progress under microscope every -five minutes.

Once cells are just beginning to lift off (˜10 minutes), triturate wells repeatedly to dislodge cells.

Once cells are dislodged, return the plate to the incubator for another three-five minutes.

Lightly triturate cells before transferring to 50 mL conical.

For 0.25% Trypsin in Versene:

Add 0.25% Trypsin to each well at half the volume denoted on TABLE 2. Incubate cells at 37° C. for 3-6 minutes, or until cells appear to dislodge from the bottom of the plate, as observed under a microscope.

Detach cells from well by triturating repeatedly. Check under microscope to ensure mostly single cells.

Transfer cells to 50 mL conical containing an equal volume of stop solution.

Wash wells with stop solution to collect any remaining cells. Collect in 50 mL conical.

Spin down cells at 300 g for five minutes.

Remove CMs from centrifuge, aspirate supernatant, and resuspend pellet in 24 mL replate solution, triturating enough times to break up pellet into small clusters.

Aspirate Matrigel from plate and divide cell suspension evenly into each well. Rock plate gently to evenly distribute cells.

The following day, aspirate spent media, rinse with DPBS, and feed with RPMI/B27.

Feed cells every other day with RPMI/B27 until lactate purification on day 20.

Note: Cells should resume beating two-three days following a harvest. Ensure that cells have begun beating prior to lactate purification.

### Step 5: Lactate Purification

Wash wells with DPBS and feed with DM EM (no glucose, no sodium pyruvate)+four mM Sodium L-Lactate for metabolic-based CMs purification.

Note: CMs should be actively beating during lactate selection. Non-CMs population will lift off from the plate, driving increased CM purity.

Repeat step 1 two days later for a second round of lactate purification.

Two days later, wash wells with DPBS and feed with RPMI/B27.

Note: Do not go beyond four straight days of lactate selection without a recovery day in RPMI/B27.

Feed cells every other day with RPMI/B27 until ready for use.

### Step 6: 3D Cardiac Microtissue Generation

### Preparation:

Pipette sterile 2% (wt/vol) agarose in DPBS into 35-microwell negative molds with hemispherical bottoms. Be sure to avoid microbubbles when casting agarose molds.

Carefully remove gelled agarose hydrogels (three minutes to gel) from the negative molds.

Equilibrate agarose hydrogels in RPMI/B27 with 1% Pen-Strep for at least one hour or overnight in a 37° C. incubator.

### Generating 3D Cardiac Microtissues:

Allow three-four days for hiPSC-CMs to recover from lactate purification before generating 3D cardiac microtissues.

Harvest CMs according to protocol 4.1-4.9.

Remove CMs from centrifuge, aspirate supernatant, and resuspend pellet in 5-10 mL stop solution, triturating enough times to break up pellet into small clusters.

Take 10 μL aliquot of cell mixture and dilute 1:1 in Trypan blue (10 μL) in an Eppendorf tube. Count total hiPSC-CMs with hemacytometer.

Optional: Collect 0.5×10⁵-1.0×10⁶ cells for flow cytometry.

Calculate the number of remaining hiPSC-CMs and add 5% human cardiac fibroblast (hCF, of total mixed cell count) to the cell mixture.

Spin down cells at 300 g for five minutes.

While cells spin down, transfer equilibrated agarose hydrogels into a 24/12/6 well-plate that is compatible with a stimulator lid and an electrical pacing system. Carefully remove excess solution from the wells with a P-200 pipette.

Remove hiPSC-CM/hCF cell mixture from centrifuge, aspirate supernatant, and resuspend pellet in the appropriate volume of replate solution. Triturate sufficiently to break pellet into small clusters and for even cell distribution.

Note: Optimal seeding density for hiPSC-CM/hCF mixture is 3.5-7.0×105 cells in 100 μL of replate solution per agarose hydrogel. Higher densities may result in a necrotic core in microtissues.

Pipette 100 μL of cell mixture into recesses in the agarose hydrogels.

Allow the cells to gravity settle into individual wells for thirty minutes in 37° C. (yielding 10,000-20,000 cells/microtissue).

Gently fill the 24/12/6-well plate containing cell-seeded agarose hydrogels with replate media. Ensure that agarose hydrogels are complete submerged with media.

Carefully place stimulation lids to 24/12/6-well plates and connect to an electrical pacing system to electrically field stimulate the cell-seeded agarose hydrogels with a 1 Hz, 10.0-15.0 V, and 4.0 milliseconds duration bipolar pulse train in a 37° C. incubator.

The following day, carefully remove spent media with 10 mL pipettes and feed with RPMI/B27+10% FBS+1% Pen-Strep.

Feed microtissues with RPMI/B27+10% FBS+1% Pen-Strep every other day and continue to electrically stimulate cells until ready for downstream experiments.

Note: Electrical stimulation will aid with the self-assembly and electrical maturation of microtissues. Significant compaction will occur within the first three days (see FIG. 6A), and microtissues will begin to beat 2-4 days post microtissue formation.

### EXAMPLE 2

### Platform for Atrial Arrhythmia Risk Assessment

Cardiomyocyte subtype, maturation state, and structural organization influence spontaneous beating rates. Retinoic acid supplementation at days 3-6 of differentiation yielded cardiomyocytes with significantly reduced MLC2v expression compared to those without retinoic acid supplementation (MLC2v+_{aCM}=18±5 vs. MLC2v+_{vCM}=31±0.2, p<0.05; FIG. 6), indicating successful subtype specification. The inventors consistently generated high-purity cardiomyocytes following metabolic-based lactate purification for both atrial and ventricular subtypes (cTnT+_{aCM}=83±5% vs. cTnT+_{vCM}=89±5%, p>0.05; FIG. 6). Fluorescence imaging of calcium transients in 2D monolayer cultures without electrical stimulation showed that spontaneous activity varied with cardiomyocyte subtypes, with atrial cardiomyocytes demonstrating faster automaticity than ventricular cardiomyocytes at day 28 of differentiation (freq_{aCM,D28}=0.64±0.25 Hz vs. freq_{vCM,D28}=0.31±0.06 Hz, p<0.05; FIG. 2A and FIG. 2B).

Interestingly, in a small batch of differentiation where the inventors cultured cardiomyocytes for up to forty-five days, we measured a significant increase in spontaneous beating rates in atrial cardiomyocytes to 1.72±0.49 Hz (p<0.0001 compared to day 28), while that of ventricular cardiomyocytes remained relatively unchanged 0.53±0.28 Hz (p=0.24 compared to day 28). These differences resulted in rate-dependent calcium transients prolongation in ventricular cardiomyocytes (CaT_{aCM}=0.94±0.18 seconds vs. CaT_{vCM}=1.34±0.17 sec, p<0.001, FIG. 2D), although the calcium transients of ventricular cardiomyocytes demonstrated wider peaks with a prominent plateau phase (black arrows, FIG. 2A). The inventors noted a decrease in spontaneous activity following 3D microtissue formation with 5% human cardiac fibroblasts. The frequency of spontaneous activity significantly decreased to 0.41±0.13 Hz (p<0.05 and p<0.0001 compared to monolayer culture at day 28 and day 45, respectively) in 3D atrial microtissues, while spontaneous activity was eliminated in 3D ventricular microtissues (FIG. 2C). Thus, the subtype, maturation state, and structural organization of cardiomyocytes modulate their electrophysiological behavior.

The 3D atrial microtissues are characterized by longer rise time to peak and shorter action potential duration compared to 3D ventricular microtissues. Using optical mapping, the inventors were able to isolate highly repeatable action potential traces from 3D cardiac microtissues under 1 Hz pacing with minimal beat-to-beat and microtissue variability within the same batch, although greater variations in ventricular action potential traces were observed during late repolarization (FIG. 7A-D). While batch-to-batch differences in action potential durations were observed, these variations were small and remained distinct between atrial and ventricular microtissues (FIG. 7E-F). Atrial microtissues demonstrated slower rise time to peak. They lacked a prominent plateau phase, resulting in APD₃₀, APD₅₀, APD₈₀, and APD_{MxR} values that were on average 3x shorter than that of ventricular microtissues (FIG. 3A-F). Only APD_{Tri}, a measure of action potential triangulation during late repolarization calculated as the difference between APD_{MxR} and APD₅₀, did not present statistical differences between atrial and ventricular microtissues (FIG. 3G), suggesting that differences in action potential shape primarily arose from ion channel/currents that were responsible for early repolarization (Iₜₒ, I_{Kur}) and the plateau phase (I_{CaL}). TABLE 3 summarizes the chamber-specific differences in action potential parameters measured via optical mapping in the 3D microtissues. The inventors observed a sharp action potential peak during early repolarization (black arrow, FIG. 3A) in 3D microtissues generated from atrial cardiomyocytes cultured for forty-five days, Thus, the inventors could mature the input cardiomyocytes to construct microtissues with more well-developed ion channels including the Iₜₒ channel, which is a signature of more adult-like atrial action potential.

TABLE 3. Atrial and ventricular action potential differences. A TABLE summarizing action potential differences between cardiomyocyte subtypes. Data are presented as mean±standard deviation.

TABLE 3
Atrial and ventricular action potential differences

| AP parameter | Atrial | Ventricular | Statistics |
|---|---|---|---|
| Rise Time (ms) | 11.7 ± 3.0 | 7.6 ± 1.6 | p < 0.001 |
| APD₃₀ (ms) | 76.6 ± 7.5 | 266.1 ± 39.4 | p < 0.001 |
| APD₅₀ (ms) | 97.8 ± 8.3 | 314.7 ± 43 | p < 0.001 |
| APD₈₀ (ms) | 124.8 ± 10.8 | 355.5 ± 47.2 | p < 0.001 |
| APD_{MxR} (ms) | 126.2 ± 11.8 | 341.9 ± 45.1 | p < 0.001 |
| APD_{Tri} (ms) | 28.5 ± 7.9 | 27.2 ± 4.8 | p = 0.297 |

3D microtissues exhibit dose-dependent and chamber-specific responses to known I_{Kur} and i_{f} channels blockers. The inventors tested the 3D cardiac microtissues with 4-aminopyridine, a drug which more sensitively targets the atrial-specific I_{Kur} channel at low doses while targeting Iₜₒ at higher doses, to investigate if someone could recapitulate chamber-specific responses. Burashnikov & Antzelevitch. J. Atr. Fibrillation, 1, 98-107 (2008). The inventors identified a dose-dependent response to 4-aminopyridine in atrial microtissues that was absent in ventricular microtissues (FIG. 4A-B). APD₃₀, APD₅₀, APD₈₀, and APD_{MxR} in atrial microtissues were all significantly prolonged with increasing doses of 4-aminopyridine (FIG. 8), clearly visualized as a rightward shift in the cumulative probability distribution shown for APD₈₀ in FIG. 4A. While not observed across all doses, the inventors also found a significant increase in rise time and APD_{Tri} in the atrial microtissues at the 100 μM 4-aminopyridine dose (FIG. 8). No significant changes in APD₃₀, APD₅₀, APD₈₀, and APD_{MxR} in the ventricular microtissues were detected across all drug dosages of 4-aminopyridine, although two clusters of microtissues with distinct action potential durations (FIG. 4B). These clusters were likely reflective of the larger variations observed in late repolarization between the action potential traces of the ventricular cardiomyocyte microtissues (FIG. 7D).

The inventors also tested the effect of ivabradine, an I_{f} blocker, on the spontaneous action potential activity of atrial microtissues. As shown in the middle panel of FIG. 4C, a 1 μM dose of ivabradine was sufficient to eliminate spontaneous activity in more than half of the atrial microtissues. In the few remaining atrial microtissues with spontaneous activity, many did not undergo changes in action potential properties or cycle length, suggesting that these samples may not have respond to the treatment. A small subset showed either a single spontaneous beat or spontaneous activity with increased cycle length. Inspection of the voltage traces showed that these events appeared to have been driven by a series of failed depolarizations, as drifting baseline potential failed to reach the necessary threshold to produce successful depolarization (blue arrow, FIG. 4C, left panel). These “apparent” cycle length due to failed depolarization are reported as blue squares in the right panel of FIG. 4C.

Computational modeling of chamber specific hiPSC-cardiomyocytes recapitulates the findings. The inventors incorporated the atrial-specific I_{Kur} channel into a previously established hiPSC-cardiomyocyte action potential computational model. Paci et al., Ann. Biomed. Eng., 41, 2334-2348 (2013) and Br. J. Pharmacol., 172, 5147-5160 (2015). The inventors adjusted the maximum conductance values for the major ion channels, summarized in TABLE 1, to match the action potential shape of the atrial and ventricular microtissues (FIG. 5A-B). The best-fitted parameters showed that atrial microtissues had reduced I_{Na} and I_{CaL}when compared to ventricular microtissues (FIG. 5C), explaining the slower rise time to peak and lack of a well-developed plateau phase in the experimentally measured atrial action potential traces. Atrial microtissues had reduced I_{Kr}, I_{Ks}, and I_{K1} but increased Iₜₒ and I_{f} when compared to the ventricular microtissues (FIG. 5C), which together accounted for the narrowed action potential peak and “positively drifting” resting membrane potential that resulted in increased spontaneous activity in the 3D atrial microtissues. To explore the effects of I_{Kur} on the action potential shape and duration of atrial microtissues, we conducted a large g_{Kur} sweep, relative to the optimized g_{Kur} parameter in the atrial model, and reported the corresponding changes in APD₃₀, APD₅₀, and APD₈₀ in FIG. 5D, with action potential waveforms for representative g_{Kur} values presented in FIG. 5E. As expected, the observed changes in action potential duration followed a behavior that resembled a dose-response curve, with decreased conductances, reflective of drug-induced I_{Kur} block, resulting in action potential duration prolongation. FIG. 5E also showed that changes in I_{Kur} altered the concavity of the action potential repolarization phase.

The inventors performed a sigmoidal fit on the action potential duration response curve and used the modeled equation to correlate how drug-induced action potential duration prolongation by 4-aminopyridine correspond to changes in g_{Kur} in the model.

### EXAMPLE 3

### Fibroblast Staining of 3D Microtissues

The inventors obtained representative photographic images of a 3D cardiac microtissue stained with cardiac troponin I (cTnI), vimentin, and Hoechst. The images confirmed the presence of a low percentage of fibroblasts distributed throughout the microtissue. Immunohistochemical staining on monolayer cultures of either cardiomyocytes or fibroblasts with both cTnI and vimentin confirmed that input hiPSC-cardiomyocytes positively stain for cTnI, as observed by striations on the yellow inset while staining negative for vimentin.

### EXAMPLE 4

### Additional TABLES.

TABLE 4
TaqMan gene expression assays for RT-qPCR

| Marker | Gene | Target | TaqMan Assay |
|---|---|---|---|
| Cardiac | MYL2 | Ventricular | H500160403_m1 |
| phenotype | IRX4 | Ventricular | Hs00212560_m1 |
| | NR2F2 | Atrial | Hs00818842_m1 |
| | NPPA | Atrial | Hs00383230 g1 |
| Ion | SCN5A | I_{Na} | Hs00165693_m1 |
| channels | CACNA1C | I_{CaL} | Hs00167681_m1 |
| | CACNA1D | I_{CaL} (atrial | Hs00167753_m1 |
| | | subunit) | |
| | KCND3 | Iₜₒ | Hs00542597_m1 |
| | KCNA5 | I_{KUr} | Hs00969279_s1 |
| | KCNH2 | I_{Kr} | Hs00165120_m1 |
| | KCNQT | I_{Ks} | Hs00923522_m1 |
| | KCNJ2 | I_{Kf} | Hs00265315_m1 |
| | KCNJ3 | I_{KAch} | HsM4334861_s1 |
| | HCN4 | I_{f} | Hs00923522 m1 |

TABLE 4 shows TaqMan gene expression assays for RT-qPCR. TaqMan Assay probes and their target genes used for gene expression assays via RT-qPCR.

TABLE 5
4-Aminopyridine action potential drug response

| Cell type | Parameter | Baseline | 1 μM |
|---|---|---|---|
| Atrial | Rise Time (ms) | 12.2 ± 2.4 | 11.9 ± 2.6 |
| | APD₃₀ (ms) | 70.7 ± 9.5 | 75.2 ± 11.4* |
| | APD₅₀ (ms) | 90.1 ± 11.7 | 95.7 ± 12.9** |
| | APD₈₀ (ms) | 114.6 ± 15.5 | 121.4 ± 16.2** |
| | APD_{MxR} (ms) | 118.3 ± 14.3 | 126.6 ± 15.8*** |
| | APD_{Tri}/APD_{MxR} | 0.24 ± 0.04 | 0.24 ± 0.03 |
| Cell type | Parameter | 3 μM | 100 μM |
| Atrial | Rise Time (ms) | 11.8 ± 2.2 | 14.0 + 3.7**** |
| | APD₃₀ (ms) | 75.2 ± 11.4* | 84.0 ± 10.6**** |
| | APD₅₀ (ms) | 97.5 ± 11.4*** | 104.8 ± 12.0**** |
| | APD₈₀ (ms) | 123.5 ± 14.7*** | 129.9 ± 15.9**** |
| | APD_{MxR} (ms) | 128.3 ± 12.9**** | 136.6 ± 14.6**** |
| | APD_{Tri}/APD_{MxR} | 0.24 ± 0.03 | 0.23 ± 0.03 |
| Cell type | Parameter | Baseline | 1 μM |
| Ventricular | Rise Time (ms) | 6.3 ± 1.8 | 6.9 ± 1.9 |
| | APD₃₀ (ms) | 247.8 ± 29.4 | 244.6 ± 38.4 |
| | APD₅₀ (ms) | 289.6 ± 33.1 | 287.8 ± 49.9 |
| | APD₈₀ (ms) | 334.6 ± 39.8 | 329.6 ± 50.6 |
| | APD_{MxR} (ms) | 317.7 ± 35.6 | 317.7 ‡55.4 |
| | APD_{Tri}/APD_{MxR} | 0.08 ± 0.01 | 0.09 ± 0.01′ |
| Cell type | Parameter | 3 μM | 100 μM |
| Ventricular | Rise Time (ms) | 7.1 ± 2.4* | 7.5 ± 2.6** |
| | APD₃₀ (ms) | 233.3 ± 35.8*** | 236.7 ± 32.5* |
| | APD₅₀ (ms) | 275.7 ± 47.7** | 280.0 ± 44.0* |
| | APD₈₀ (ms) | 321.6 ± 54.1 | 325.6 ± 48.5 |
| | APD_{MxR} (ms) | 307.5 ± 55.2* | 312.5 ± 51.7 |
| | APD_{Tri}/APD_{MxR} | 0.10 ± 0.02**** | 0.10 ± 0.02**** |

TABLE 5 discloses 4-aminopyridine (4-AP) action potential drug response. Averaged data for six action potential metrics of atrial and ventricular microtissues in response to different dosages of 4-aminopyridine. Values are presented as mean±standard deviation and represent data from n=3 differentiation batches.

TABLE 6
CT values for gene expression analysis

| Marker | Gene | Ventricular | Atrial | p-values |
|---|---|---|---|---|
| Cardiac | MYL2 | 12.1 ± 1.7 | 18.8 ± 2.6 | *p = 0.03 |
| phenotype | IRX4 | 14.8 ± 0.2 | 17.1 ± 0.9 | *p = 0.04 |
| | NR2F2 | 18.0 ± 1.5 | 14.0 ± 0.3 | **p < 0.01 |
| | NPPA | 13.7 ± 0.5 | 10.2 ± 0.6 | **p < 0.01 |
| Ion | SCN5A | 16.4 ± 0.9 | 14.9 ± 0.2 | p = 0.06 |
| channels | CACNA1C | 14.6 ± 0.6 | 14.7 ± 0.7 | p = 0.92 |
| | CACNA1D | 14.6 ± 0.6 | 17.3 ± 0.4 | *p = 0.03 |
| | KCND3 | 23.2 ± 0.8 | 23.3 ± 1.2 | p = 0.93 |
| | KCNA5 | 21.8 ± 0.2 | 16.5 ± 0.5 | **p < 0.01 |
| | KCNH2 | 15.5 ± 0.1 | 15.5 ± 0.4 | p = 0.96 |
| | KCNQ1 | 15.0 ± 0.6 | 14.8 ± 0.4 | p = 0.72 |
| | KCNJ2 | 21.4 ± 0.8 | 25.3 ± 2.6 | p = 0.08 |
| | KCNJ3 | 16.3 ± 0.5 | 14.4 ± 0.9 | *p = 0.05 |
| | HCN4 | 13.1 ± 0.3 | 12.3 ± 1.0 | p = 0.29 |

TABLE 6 shows dCT values from gene expression analysis. dCT values, relative to the housekeeping gene 18S, obtained from gene expression analysis of ventricular and atrial microtissues with p-values highlighting differences between the two cardiac subtypes. Values are presented as mean±standard deviation and represent data from n=3 differentiation batches.

### LIST OF EMBODIMENTS

Specific compositions and methods of the manufacture and use of a platform for atrial arrhythmia risk assessment have been described. The scope of the invention should be defined solely by the claims. A person having ordinary skill in the biomedical art will interpret all claim terms in the broadest possible manner consistent with the context and the spirit of the disclosure. The detailed description in this specification is illustrative and not restrictive or exhaustive. This invention is not limited to the particular methodology, protocols, and reagents described in this specification and can vary in practice. When the specification or claims recite ordered steps or functions, alternative embodiments might perform their functions in a different order or substantially concurrently. Other equivalents and modifications besides those already described are possible without departing from the inventive concepts described in this specification, as persons having ordinary skill in the biomedical art recognize.

All patents and publications cited throughout this specification are incorporated by reference to disclose and describe the materials and methods used with the technologies described in this specification. The patents and publications are provided solely for their disclosure before the filing date of this specification. All statements about the patents and publications' disclosures and publication dates are from the inventors' information and belief. The inventors make no admission about the correctness of the contents or dates of these documents. Should there be a discrepancy between a date provided in this specification and the actual publication date, then the actual publication date shall control. The inventors may antedate such disclosure because of prior invention or another reason. Should there be a discrepancy between the scientific or technical teaching of a previous patent or publication and this specification, then the teaching of this specification and these claims shall control.

When the specification provides a range of values, each intervening value between the upper and lower limit of that range is within the range of values unless the context dictates otherwise.

### CITATION LIST

A person having ordinary skill in the biomedical art can use these patents, patent applications, and scientific references as guidance to predictable results when making and using the invention.

### Patent Literature

WO 2020/113025 A1 (Milica Radisic) “Methods for tissue generation.” One aspect of the specification relates to an ex vivo tissue system comprising a chamber-specific cardiac tissue and a bioreactor, wherein the bioreactor includes at least two elastic sensing elements configured to support the chamber-specific cardiac tissue. The Biowire II platform enables the production of high-fidelity 3D human cardiac tissues from many different cell sources. In some embodiments, the POMaC polymer wires in the platform are used as both a mechanical stimulus attachment point for the tissue and a force sensor, enabling simultaneous assessments of intracellular calcium fluctuations and contractile force. Using heart chamber-specific directed differentiation and electrical conditioning protocols, cardiac tissues with distinct atrial or ventricular phenotypes as well as a combined heteropolar atrio-ventricular tissues are produced, demonstrating the utility of these preparations for drug testing.

### Non-Patent Literature

Abdelnabi et al., Ivabradine and AF: Coincidence, correlation or a new treatment? Arrhythm. Electrophysiol. Rev., 8, 300-303 (2020).

Amos et al., Differences between outward currents of human atrial and subepicardial ventricular myocytes. J. Physiol. 491(Pt 1), 31-50 (1996).

Antzelevitch et al. Annals N.Y. Acad. Sci. (2010) describes atrial and ventricular channels.

Benzoni et al., Human iPSC modelling of a familial form of atrial fibrillation reveals a gain of function of I_{f} and I_{CaL} in patient-derived cardiomyocytes. Cardiovasc. Res., 116, 1147-1160 (2020).

Branco et al., Transcriptomic analysis of 3D cardiac differentiation of human induced pluripotent stem cells reveals faster cardiomyocyte maturation compared to 2D culture. Sci. Reprts, 9, 9229 (2019).

Burashnikov & Antzelevitch. How do atrial-selective drugs differ from antiarrhythmic drugs currently used in the treatment of atrial fibrillation? J. Atr. Fibrillation, 1, 98-107 (2008).

Burridge et al., Chemically defined generation of human cardiomyocytes. Nature Methods, 11, 855-860 (2014) describes cardiac-directed differentiation.

Centurion, Atrial fibrillation in the Wolff-Parkinson-White Syndrome. J. Atr. Fibrillation, 4, 287 (2011).

Chauveau et al., Induced pluripotent stem cell-derived cardiomyocytes provide in vivo biological pacemaker function. Circ. Arrhythm. Electrophysiol. (2017).

Clerx et al., Four Ways to fit an ion channel model. Biophys. Journal, 117, 2420-2437 (2019).

Colilla et al., Estimates of current and future incidence and prevalence of atrial fibrillation in the U.S. adult population. Am. J. Cardiol., 112, 1142-1147 (2013).

Cyganek et al., Deep phenotyping of human induced pluripotent stem cell-derived atrial and ventricular cardiomyocytes. JCI Insight, 3 (2018) describes cardiac-directed differentiation.

Dan & Dobrev, Antiarrhythmic drugs for atrial fibrillation: Imminent impulses are emerging. Int. J. Cardiol. Heart Vasc., 21, 11-15 (2018).

Ferdinandy et al., Eur Heart Journal (2019) describes diseased myocardium with impaired I_{Ks}/I_{K1} function and repolarization reserve.

Garg et al., Human induced pluripotent stem cell-derived cardiomyocytes as models for cardiac channelopathies: A primer for non-electrophysiologists. Circ. Res. 123, 224-243 (2018).

Gaztanaga et al., Time to recurrence of atrial fibrillation influences outcome following catheter ablation. Heart Rhythm, 10, 2-9 (2013).

Geng, Lin, & Nguyen, Revisiting antiarrhythmic drug therapy for atrial fibrillation: Reviewing lessons learned and redefining therapeutic paradigms. Front. Pharmacol., 11, 581837 (2020).

Giacomelli et al., Three-dimensional cardiac microtissues composed of cardiomyocytes and endothelial cells co-differentiated from human pluripotent stem cells. Development, 144,1008-1017 (2017).

Giacomelli et al., Human-iPSC-derived cardiac stromal cells enhance maturation in 3D cardiac microtissues and reveal non-cardiomyocyte contributions to heart disease. Cell Stem Cell, 26, 862-879 (2020).

Gintant et al., Use of human induced pluripotent stem cell-derived cardiomyocytes in preclinical cancer drug cardiotoxicity testing: A scientific statement from the American Heart Association. Circ. Res., 125, e75-e92 (2019).

Goldfracht et al., Generating ring-shaped engineered heart tissues from ventricular and atrial human pluripotent stem cell-derived cardiomyocytes. Nature Commun., 11, 75 (2020).

Grandi et al., Human atrial action potential and Ca2+ model: sinus rhythm and chronic atrial fibrillation. Circ. Res., 109, 1055-1066 (2011).

Gunawan et al., Drug screening platform using human induced pluripotent stem cell-derived atrial cardiomyocytes and optical mapping. Stem Cells Trans!. Med., 10, 68-82 (2021).

Hanley, Robinson, & Kowey, Status of antiarrhythmic drug development for atrial fibrillation: New drugs and new molecular mechanisms. Circ. Arrhythm. Electrophysiol., 9, e002479 (2016).

Hong et al., Spiral reentry waves in confluent layer of HL-1 cardiomyocyte cell lines. Biochem. Biophys. Res. Commun., 377, 1269-1273 (2008).

Jacquemet & Henriquez, Modelling cardiac fibroblasts: interactions with myocytes and their impact on impulse propagation. Europace, 9 Suppl 6, vi29-37 (2007).

Kim et al., Mechanism of automaticity in cardiomyocytes derived from human induced pluripotent stem cells. J. Mol. Cell. Cardiol., 81, 81-93 (2015).

Kim et al., Directed fusion of cardiac spheroids into larger heterocellular microtissues enables investigation of cardiac action potential propagation via cardiac fibroblasts. PLoS One, 13, e0196714 (2018).

Klesen et al., Cardiac fibroblasts: active players in (atrial) electrophysiology? Herzschrittmacherther. Elektrophysiol. 29:62-69, 2018.

Kofron et al., Gq-activated fibroblasts induce cardiomyocyte action potential prolongation and automaticity in a three-dimensional microtissue environment. Am. J. Physiol. Heart Circ. Physiol., 313, H810—H827 (2017).

Kofron et al., A predictive in vitro risk assessment platform for pro-arrhythmic toxicity using human 3D cardiac microtissues. Sci. Reports, 11(1),10228 (2021).

Kornej et al., Epidemiology of atrial fibrillation in the 21st Century: Novel methods and new insights. Circ. Res., 127, 4-20 (2020).

Lee et al., Human pluripotent stem cell-derived atrial and ventricular cardiomyocytes develop from distinct mesoderm populations. Cell Stem Cell, 21, 179e174-194e174 (2017).

Lemme et al., Atrial-like engineered heart tissue: an in vitro model of the human atrium. Stem Cell Reports, 11,1378-1390 (2018).

Lian et al., Directed cardiomyocyte differentiation from human pluripotent stem cells by modulating Wnt/β-catenin signaling under fully defined conditions, Nature Protocols, 8(1),162-75 (January 2013).

Lian et al., Efficient differentiation of human pluripotent stem cells to endothelial progenitors via small-molecule activation of WNT signaling. Stem Cell Reports, 3, 804-816 (2014).

Livak & Schmittgen. Analysis of relative gene expression data using real-time quantitative PCR and the 2(-delta C(T)) method. Methods, 25, 402-408 (2001).

Laverty et al. British J. Pharmacol. (2011) describe toxicities leading to drug withdrawal during development and from the global market.

Ma et al., High purity human-induced pluripotent stem cell-derived cardiomyocytes: electrophysiological properties of action potentials and ionic currents. Am. J Physiol. Heart Circ. Physiol., 301, H2006-2017 (2011).

Maleckar et al., K+ current changes account for the rate dependence of the action potential in the human atrial myocyte. Am. J. Physiol. Heart Circ. Physiol., 297, H1398-1410 (2009).

Morillo et al., Atrial fibrillation: the current epidemic. J. Geriatr. Cardiol ,14, 195-203 (2017).

Mujovic et al., Catheter ablation of atrial fibrillation: An overview for clinicians. Adv. Ther., 34, 1897-1917 (2017).

Nattel et al., Dose-dependence of 4-aminopyridine plasma concentrations and electrophysiological effects in dogs: Potential relevance to ionic mechanisms in vivo. Circulation, 101, 1179-1184 (2000).

Navarrete et al., Screening drug-induced arrhythmia [corrected] using human induced pluripotent stem cell-derived cardiomyocytes and low-impedance microelectrode arrays. Circulation. 128, S3-S13 (2013).

Noireaud, & Andriantsitohaina. Recent insights in the paracrine modulation of cardiomyocyte contractility by cardiac endothelial cells. Biomed. Res. Int., 2014, 923805 (2014).

Paci, Hyttinen, Aalto-Setala, & Severi, Computational models of ventricular- and atrial-like human induced pluripotent stem cell derived cardiomyocytes. Ann. Biomed. Eng., 41, 2334-2348 (2013).

Paci, Hyttinen, Rodriguez, & Severi, Human-induced pluripotent stem cell-derived versus adult cardiomyocytes: An in silico electrophysiological study on effects of ionic current block. British J. Pharmacol., 172, 5147-5160 (2015).

Paik, Chandy, & Wu, Patient and disease-specific induced pluripotent stem cells for discovery of personalized cardiovascular drugs and therapeutics. Pharmacol. Rev., 72, 320-342 (2020).

Pang et al., Workshop report: FDA workshop on improving cardiotoxicity assessment with human-relevant platforms. Circ. Res., 125, 855-867 (2019).

Pei et al., Chemical-defined and albumin-free generation of human atrial and ventricular myocytes from human pluripotent stem cells. Stem Cell Res 19: 94-103 (2017).

Plotnikov et al., Xenografted adult human mesenchymal stem cells provide a platform for sustained biological pacemaker function in canine heart. Circulation. 116, 706-713 (2007).

Radisic et al., Functional assembly of engineered myocardium by electrical stimulation of cardiac myocytes cultured on scaffolds. Proc. Natl. Acad. Sci., U.S.A., 101, 18129-18134 (2004).

Ravens & Wettwer, Ultra-rapid delayed rectifier channels: molecular basis and therapeutic implications. Cardiovasc. Res., 89, 776-785 (2011).

Ridley et al., Inhibition of HERG K+ current and prolongation of the guinea-pig ventricular action potential by 4-aminopyridine. J. Physiol., 549, 667-672 (2003).

Rottner et al., Catheter ablation of atrial fibrillation: State of the art and future perspectives. Cardiol. Ther., 9, 45-58 (2020).

Rupert, Kim, Choi, & Coulombe, Human cardiac fibroblast number and activation state modulate electromechanical function of hiPSC-cardiomyocytes in engineered myocardium. Stem Cells Int., 2020, 9363809 (2020).

Sacchetto et al., Modeling Cardiovascular Diseases with hiPSC-Derived Cardiomyocytes in 2D and 3D Cultures. Int. J. Mol. Sci., 21 (2020).

Schram, Pourrier, Melnyk, & Nattel, Differential distribution of cardiac ion channel expression as a basis for regional specialization in electrical function. Circ. Res., 90, 939-950 (2002).

Sinnecker, Laugwitz, & Moretti, Induced pluripotent stem cell-derived cardiomyocytes for drug development and toxicity testing. Pharmacol. Ther., 143, 246-252 (2014).

Soepriatna, et al. Human Atrial cardiac microtissues for chamber-specific arrhythmic risk assessment. Cell Mol. Bioeng., 14, 441-457 (2021).

Tamargo, Caballero, Gomez, & Delpon, 1(Kur)/Kv1.5 channel blockers for the treatment of atrial fibrillation. Expert Opin. Investig, Drugs, 18, 399-416 (2009).

Tanner & Beeton, Differences in ion channel phenotype and function between humans and animal models. Front. Biosci. (Landmark ed.), 23, 43-64 (2018).

Tohyama et al., Distinct metabolic flow enables large-scale purification of mouse and human pluripotent stem cell-derived cardiomyocytes. Cell Stem Cell, 12, 127-137 (2013).

Vasquez, Benamer, & Morley, The cardiac fibroblast: Functional and electrophysiological considerations in healthy and diseased hearts. J. Cardiovasc. Pharmacol., 57, 380-388 (2011).

Walden, Dibb, & Trafford, Differences in intracellular calcium homeostasis between atrial and ventricular myocytes. J. Mol. Cell Cardiol., 46, 463-473 (2009).

Wijesurendra & Casadei. Mechanisms of atrial fibrillation. Heart, 105, 1860-1867 (2019).

Wilke et al. Nature Rev. Drug Discov. (2007) describes toxicities leading to drug withdrawal from the United States market.

Woods & Olgin, Atrial fibrillation therapy now and in the future: Drugs, biologicals, and ablation. Circ. Res., 114, 1532-1546 (2014).

Xie et al., So little source, so much sink: Requirements for afterdepolarizations to propagate in tissue. Biophys. J., 99, 1408-1415 (2010).

Zhang, Su, & Mende, Cross talk between cardiac myocytes and fibroblasts: from multiscale investigative approaches to mechanisms and functional consequences. Am. J. Physiol. Heart Circ. Physiol., 303, H1385-1396 (2012).

Zhao et al., A platform for generation of chamber-specific cardiac tissues and disease modeling, Cell, 176(4), 913-927, E18 (Feb. 7, 2019). This publication discloses a platform that enables creation of electrophysiologically distinct atrial and ventricular tissues, that can provide months long biophysical stimulation of 3D tissues to model a polygenic disease. By combining directed cell differentiation with electrical field conditioning, the authors engineered electrophysiologically distinct atrial and ventricular tissues with chamber-specific drug responses and gene expression.

Zhou & Pu, Recounting cardiac cellular composition. Circ. Res. 118, 368-370 (2016).

### TEXTBOOKS AND TECHNICAL REFERENCES

Current Protocols in Immunology (CPI). Coligan et al., eds. (John Wiley and Sons, Inc., 2003).

Current Protocols in Molecular Biology (CPMB). Ausubel, ed. (John Wiley and Sons, 2014).

Current Protocols in Protein Science (CPPS), Coligan, ed. (John Wiley and Sons, Inc., 2005).

Janeway's Immunobiology 9th edition, Murphy et al., eds. (Taylor & Francis Limited, 2017).

Laboratory Methods in Enzymology: DNA. Lorsch, ed. (Elsevier, 2013).

Lewin's Genes XII. Krebs et al., eds. (Jones & Bartlett Publishers (2014).

Luttmann et al., Immunology (Elsevier, 2006).

Molecular Cloning: A Laboratory Manual, 4th edition, Green & Sambrook, eds. (Cold Spring Harbor Laboratory Press, Cold Spring Harbor, N.Y., USA, 2012).

Pharmaceutical Sciences 23^{rd} edition (Elsevier, 2020).

The Encyclopedia of Molecular Cell Biology and Molecular Medicine, 2^{nd} edition. Meyers, ed. (Wiley-Blackwell, 2004).

The Merck Manual of Diagnosis and Therapy, 19^{th} edition (Merck Sharp & Dohme Corp., 2018).

All patents and publications cited throughout this specification are expressly incorporated by reference to disclose and describe the materials and methods that might be used with the technologies described in this specification. The publications discussed are provided solely for their disclosure before the filing date. They should not be construed as an admission that the inventors may not antedate such disclosure under prior invention or for any other reason. If there is an apparent discrepancy between a previous patent or publication and the description provided in this specification, the present specification (including any definitions) and claims shall control. All statements as to the date or representation as to the contents of these documents are based on the information available to the applicants and constitute no admission as to the correctness of the dates or contents of these documents. The dates of publication provided in this specification may differ from the actual publication dates. If there is an apparent discrepancy between a publication date provided in this specification and the actual publication date supplied by the publisher, the actual publication date shall control.

1. An in vitro screening platform, comprising:
self-assembled 3D atrial and ventricular cardiac microtissues derived from hiPSCs;
wherein the microtissues comprise high-purity cardiomyocytes (>75% cTnT⁺); and
wherein the cardiomyocytes demonstrate subtype specification by MLC2v⁺; and
wherein the microtissues contain cardiac fibroblasts (5-50%).

2. The in vitro screening platform of claim 1, wherein the self-assembled 3D atrial and ventricular microtissues derived from hiPSC- cardiomyocytes are matured by culturing the microtissues in 3D microtissues under electrical stimulation.

3. The in vitro screening platform of claim 1, wherein the self-assembled 3D atrial and ventricular microtissues derived from hiPSC- cardiomyocytes contain all major cardiac ion channels.

4. The in vitro screening platform of claim 1, for use in measuring Ca²⁺ transient traces in addition to voltage signals.

5. The in vitro screening platform of claim 1 for use in measuring contractility or tissue force and mechanics.

6. The in vitro screening platform of claim 1 for use in mitochondrial or metabolic endpoint assessment.

7. A method of making an in vitro screening platform comprising differentiated atrial and ventricular cardiomyocytes (aCMs/vCMs) from GCaMP6f-expressing hiPSCs, comprising the steps of:
(a) generating self-assembling 3D atrial and ventricular microtissues from hiPSC-cardiomyocytes by Wnt modulation with or without the addition of retinoic acid;
(b) performing a metabolic-based lactate purification; and
(c) performing flow cytometry to assess purity and subtype.

8. The method of claim 7, wherein the step of Wnt modulation comprises the step of adding retinoic acid to generate atrial myocytes.

9. The method of claim 7, wherein the step of Wnt modulation comprises the step of not adding retinoic acid to generate ventricular myocytes.

10. The method of claim 7, further comprising the step of:
(c) assessing their calcium transients

10. The method of claim 7, further comprising the step of:
(d) optical mapping to characterize cardiomyocyte subtype differences in action potential properties.

11. The method of claim 7, further comprising the step of:
(c) modifying ion channel conductances from a published hiPSC-cardiomyocyte computational model to mimic action potential waveforms in these atrial and ventricular cardiomyocyte microtissues.

12. A method of using an in vitro screening platform, comprising the steps of:
(a) evaluating atrial-specific toxicity responses with high throughput; and
(b) using the platform to test general differences in toxicity responses between atrial and ventricular cardiomyocytes by testing drugs that do not only target atrial specific ion channels but via multiple mechanisms.

13. A method of analyzing data, comprising the atrial specific metrics of
(a) Beat interval between two spontaneous action potentials that measures proarrhythmic automaticity
(b) Pacemaker potential amplitude, a slow increase of resting membrane potential between the end of an action potential and the beginning of the following action potential, to measure the risks of producing ectopic beats.
(c) AP rise time that measures detected automatically between the rapid rise of membrane potential after pacemaker potential and the peak of action potential.
(d) APD₃₀ and APD₅₀ to measure the time difference between the action potential upstroke and the 30 or 50% repolarization time points, which detects propensity to formation of early afterdepolarization.
(e) APDₘₐₓ that measures the time difference between the action potential upstroke and the time point when the membrane potential hyperpolarizes to the lowest level. This measures excessive APD shortening that can facilitate reentry formation leading to atrial flutter and fibrillation.
